(19) United States
(12) Patent Application Publication
(10) Pub. No.: US 2023/0126790 A1
(43) Pub. Date: Apr. 27, 2023

(54) SYSTEM AND METHOD FOR PARTICLE THERAPY

(71) Applicant: HIL Applied Medical, Ltd., Givat Ram (IL)

(72) Inventors: Evgeny Papeer, Jerusalem (IL); Alex BESPALI, Jerusalem (IL); Sagi Brink-Danan, Jerusalem (IL); Pavel Komm, Modiin (IL); Raphael Van Roermund, Brussels (BE); Indranuj Dey, Jerusalem (IL)

(73) Assignee: HIL Applied Medical, Ltd., Jerusalem (IL)

(21) Appl. No.: 17/909,463

(22) PCT Filed: Mar. 10, 2021
(86) PCT No.: PCT/US2021/021682

§ 371 Date: Sep. 6, 2022

Related U.S. Application Data
(60) Provisional application No. 62/988,172, filed on Mar. 11, 2020.

(51) Int. Cl. A61N 5/10

(52) U.S. Cl. CPC A61N 5/1077; A61N 2005/1088

(57) ABSTRACT

Particle therapy systems and methods for treating patients are provided. In one implementation, a particle therapy system may include an interaction chamber for containing a target and an electromagnetic radiation source configured to generate a pulsed electromagnetic radiation beam of at least about 100 terawatts and at a repetition rate of at least about 20 Hz. The particle therapy system may further include optics configured to direct the pulsed electromagnetic radiation beam along a path towards a target in the interaction chamber. The particle therapy system may further include an actuator configured to cause relative movement between the target and the electromagnetic radiation beam at a speed associated with the repetition rate of the electromagnetic radiation source, to thereby vary a location of interaction of the pulsed electromagnetic radiation beam on a surface of the target and thereby cause a resultant emission from the target of at least about 3×10⁶ charged particles per pulse.

### CROSS REFERENCES TO RELATED APPLICATIONS

This application claims the benefit of priority of U.S. Provisional Pat. Application No. 62/988,172, filed on Mar. 11, 2020, which is incorporated herein by reference in its entirety.

### BACKGROUND

### I. Technical Field

The present disclosure relates generally to technology for particle therapy and, for example, to systems and methods that generate a beam of charged particles by applying energy to a target.

### II. Background Information

Commercial particle therapy centers are currently rare due to disadvantages in existing particle therapy systems, which generate particle beams by using large and costly particle accelerators. Accelerator-based systems can be massive and are not scalable. The energy requirements and maintenance costs inherent in operating an accelerator-based system are also immense. Taken together, these disadvantages lead to exorbitant construction and maintenance costs associated with particle therapy. In addition to the extravagant costs associated with accelerator-based particle beam generation, adjusting certain properties of the particle beam (e.g., the beam energy and beam flux) can be cumbersome and time-consuming in such systems. This leads to longer treatment times and low patient throughput, further increasing the cost of individual treatments as fewer patients share the cost burden. Accordingly, few particle therapy centers currently exist, and patients often receive inferior treatments due, in part, to unavailability of particle therapy.

The systems and methods of the present disclosure are directed towards improving performance of particle therapy systems. Although the embodiments disclosed herein contemplate the medical application of particle beam therapy, a person of ordinary skill in the art would understand that the novel particle beam generating methods and systems described below can be used in any application where such a particle beam is desired.

### SUMMARY

Embodiments consistent with the present disclosure provide systems, methods, and devices for particle therapy. Consistent with the disclosed embodiments, an exemplary system may generate and direct an electromagnetic radiation beam for irradiating a target to produce a beam of charged particles. The exemplary system may deliver the beam of charged particles to tumor within a patient’s body.

In one embodiment, a particle therapy system is provided. The particle therapy system may include an interaction chamber for containing a target and an electromagnetic radiation source configured to generate a pulsed electromagnetic radiation beam of at least 100 terawatts and at a repetition rate of at least 20 Hz. The particle therapy system may include optics configured to direct the pulsed electromagnetic radiation beam along a path towards a target in the interaction chamber. The particle therapy system may include at least one actuator configured to cause relative movement between the target and the electromagnetic radiation beam at a speed associated with the repetition rate of the electromagnetic radiation source, to thereby vary a location of interaction of the pulsed electromagnetic radiation beam on a surface of the target and thereby cause a resultant emission from the target of at least 3×10⁶ charged particles per pulse.

In another embodiment, a particle therapy system may include an interaction chamber configured to contain a target that emits charged particles in response to energy application. The particle therapy system may include an energy source for applying energy to the target and a magnetic beam line for directing a beam of charged particles from the target to a tumor of a patient in a manner that enables charged particles to strike the tumor at differing tumor locations. The particle therapy system may include at least one processor configured to selectively direct energy from the energy source to differing locations on the target. Additionally or alternatively, the at least one processor may selectively control a relative movement between the beam of charged particles and the patient to strike the tumor with charged particles at differing tumor locations.

In another embodiment, a particle therapy system may include an interaction chamber configured to contain a target having a surface with a plurality of regions thereon. The particle therapy system may also include at least one energy source and at least one processor. The at least one processor may be configured to cause the at least one energy source to deliver energy to the target in a manner causing formation of an electron cloud with a density of between 10¹⁵ cm⁻³ and 10²¹ cm⁻³ in a vicinity of at least some plurality of regions. The at least one processor may be configured to cause the at least one energy source to irradiate the target while the electron cloud is in the vicinity of the at least some of the plurality of regions, to thereby cause a plurality of charged particles to emanate from the target. The particle therapy system may include a beamline configured to deliver the plurality of charged particles to a patient.

In another embodiment, a particle therapy system may include an interaction chamber configured to contain a target. The particle therapy system may include at least one electromagnetic radiation source and at least one processor. The at least one processor may be configured to cause a pulsed beam of charged particles to be emitted from the target by regulating the at least one electromagnetic radiation source to irradiate the target with a plurality of pulse chains, each pulse chain including a preliminary pulse and a main pulse, wherein the preliminary pulse may exceed an energy flux threshold and has an energy flux on target of between 0.1 and 15 J/cm². The main pulse may have an intensity on target of at least 10¹⁸ w/cm², and a time separation between the preliminary pulse and the main pulse may be between 0.5 ns and 50 ns, such that during the time separation the target is free from irradiation exceeding the energy flux threshold.

In another embodiment, a particle therapy system may include an interaction chamber configured to contain a target and an electromagnetic radiation source configured to generate a pulsed electromagnetic radiation beam for irradiating the target and to thereby produce a polyenergetic particle beam containing multiple energy levels spanning at least 5 MeV. The particle therapy system may include a data interface configured to receive data associated with a treatment plan for a tumor and at least one processor configured to enable selection of a subset of the multiple energy levels that conforms with a treatment plan for the tumor. The particle therapy system may include a magnetic beamline configured to deliver to the tumor a portion of the polyenergetic particle beam associated with the selected subset of multiple energy levels.

Consistent with other disclosed embodiments, non-transitory computer-readable storage media may store program instructions, which are executed by at least one processor and perform any of the methods described herein.

The foregoing general description and the following detailed description are exemplary and explanatory only and are not restrictive of the claims.

### BRIEF DESCRIPTION OF THE DRAWINGS

The accompanying drawings, which are incorporated in and constitute a part of this disclosure, illustrate various disclosed embodiments. In the drawings:

FIG. 1 is a diagram illustrating an exemplary particle therapy system, consistent with disclosed embodiments.

FIG. 2 is a diagram illustrating an example configuration of an electromagnetic radiation source, in accordance with some embodiments of the present disclosure.

FIG. 3 is a diagram illustrating an example configuration of an interaction chamber, in accordance with some embodiments of the present disclosure.

FIGS. 4A and 4B are diagrams illustrating two configurations of a beamline, in accordance with some embodiments of the present disclosure.

FIG. 5 is a diagram illustrating an example configuration of the controller, in accordance with some embodiments of the present disclosure.

FIG. 6 is a flowchart of an exemplary particle therapy process, in accordance with some embodiments of the present disclosure.

FIG. 7 is a diagram illustrating a pulsed electromagnetic radiation beam that generates a pulsed particle beam, in accordance with some embodiments of the present disclosure.

FIGS. 8A and 8B are diagrams illustrating two configurations of an actuator causing a relative movement between an electromagnetic radiation beam and a target, in accordance with some embodiments of the present disclosure.

FIG. 9 is a flowchart of an exemplary particle therapy process, in accordance with some embodiments of the present disclosure.

FIG. 10 is a diagram illustrating a pulsed electromagnetic radiation beam that generates two bunches of charged particles that hit a tumor, in accordance with some embodiments of the present disclosure.

FIG. 11A is a diagram illustrating plurality of pristine Bragg peaks of an energy loss profiles as a function of depth, in accordance with some embodiments of the present disclosure.

FIG. 11B is a diagram illustrating the energy profile of an exemplary bunch of charged particles, in accordance with some embodiments of the present disclosure.

FIG. 12 is a flowchart of an exemplary particle treatment process in accordance with some embodiments of the present disclosure.

FIG. 13 is a diagrammatic illustration of an example process for causing a plurality of charged particles to emanate from a target, in accordance with some embodiments of the present disclosure.

FIG. 14A is a diagram illustrating the density of an electron cloud generated when a first dose of energy hits a target, in accordance with some embodiments of the present disclosure.

FIG. 14B is a diagram illustrating the changes in the density of the electron cloud over time, in accordance with some embodiments of the present disclosure.

FIG. 15 is a flowchart of an exemplary process for generating charged particles in accordance with some embodiments of the present disclosure.

FIG. 16 is a diagram illustrating two electromagnetic radiation sources delivering preliminary pulses and main pulses to a target, in accordance with some embodiments of the present disclosure.

FIG. 17A is a diagram illustrating the intensity of the pulses generated by at least one electromagnetic radiation source over time, in accordance with some embodiments of the present disclosure.

FIG. 17B is a diagram illustrating the energy flux of the pulses generated by at least one electromagnetic radiation source over time, in accordance with some embodiments of the present disclosure.

FIG. 18 is a flowchart of an exemplary process for generating charged particles in accordance with some embodiments of the present disclosure.

FIG. 19 is a diagram illustrating the process of selecting a subset of multiple energy levels using energy filters, in accordance with some embodiments of the present disclosure.

FIGS. 20A and 20B are a diagram illustrating different particle energy-separators, in accordance with some embodiments of the present disclosure.

FIG. 21 is a flowchart of an exemplary process for particle treatment in accordance with some embodiments of the present disclosure.

### DETAILED DESCRIPTION

The following detailed description includes references to the accompanying drawings. Wherever possible, the same reference numbers are used in the drawings and the description to refer to the same or similar parts. While several illustrative embodiments are described herein, modifications, adaptations and other implementations are possible. For example, substitutions, additions, or modifications may be made to the components illustrated in the drawings, and the illustrative methods described herein may be modified by substituting, reordering, removing, or adding steps to the disclosed methods. Accordingly, the following detailed description is not limited to the disclosed embodiments and examples. Instead, the proper scope is defined by the appended claims.

### Terms Definitions

Disclosed embodiments involve a particle therapy system. As used herein, the term “particle therapy” refers to a particle therapy medical procedure that uses a beam of particles to irradiate diseased tissue, most often in the treatment of cancer. While this description refers to this therapeutic procedure, it is to be understood that the intended scope of the innovations herein are not limited to therapy or medical procedures. Rather, it may apply any time such a particle beam is generated for any purpose. In addition, while the present disclosure generally describes a particle therapy system designed to irradiate a patient with a beam of positively charged particles (e.g., ions or protons), it may be modified easily to irradiate a patient with a beam of negatively charged particles (e.g., electrons) in the therapy medical procedure.

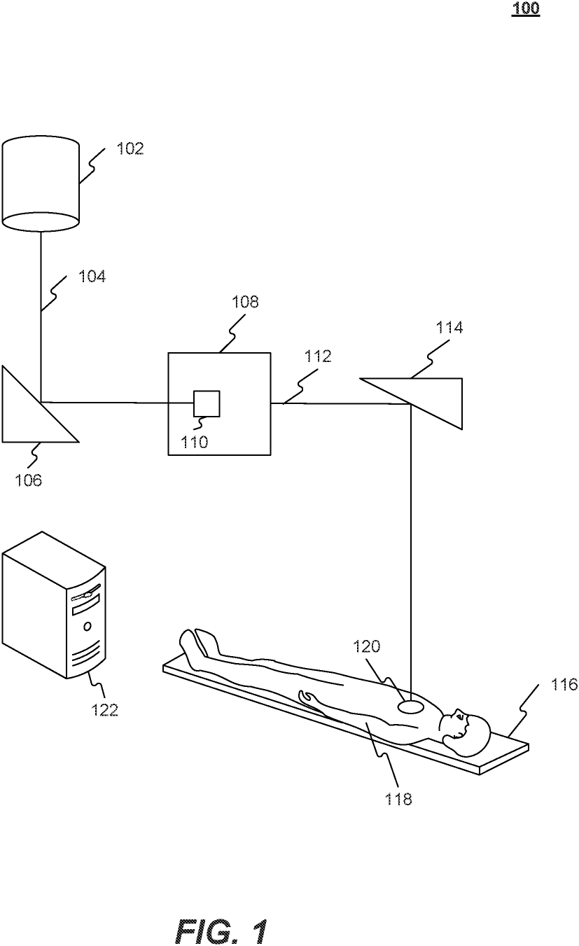

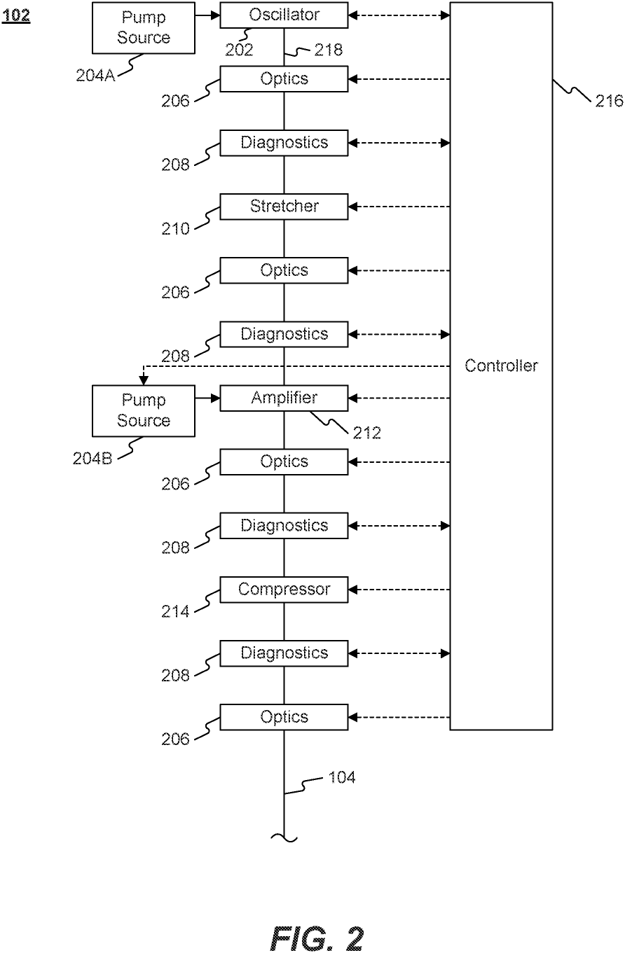

A particle therapy system in accordance with the present disclosure may include one or more sources for generating electromagnetic radiation. The term “electromagnetic radiation,” as used in the present disclosure, may refer to any form of electromagnetic radiation having any wavelength, frequency, energy, power, polarization, and/or spatial or temporal profile. In some embodiments, electromagnetic radiation may propagate in the form of a beam. For example, an electromagnetic radiation beam may be any form of electromagnetic radiation suitable for irradiating a desired location. In some embodiments, the particle therapy system may be configured to provide an electromagnetic radiation beam along a trajectory. An electromagnetic radiation beam may be configured for irradiating a target for generating charged particles (described in further detail below). As an example, an electromagnetic radiation source may provide a laser beam having traits tailored based on properties of a target. The electromagnetic radiation source may generate a pulsed electromagnetic beam to thereby cause a pulsed particle beam, or it may generate a continuous electromagnetic beam to thereby cause a continuous particle beam. Additional details on the electromagnetic radiation source are described below with reference to FIGS. 1 and 2.

A particle therapy system in accordance with the present disclosure may include optics components interacting with the electromagnetic radiation beam. As used in the present disclosure, the term “optics components,” or simply “optics,” may refer to any one or more components for controlling and manipulating an electromagnetic radiation beam in any manner, including, for example, shaping, directing, filtering, splitting, delaying, modulating, absorbing, amplifying, focusing, chopping, and/or reflecting an electromagnetic radiation beam. By way of example only, the optics components may include, light sources, lenses, mirrors, prisms, beam splitters, collimators, polarizing optics, optical modulators, optical switches, optical amplifiers, optical detectors, optical sensors, fiber optics, and/or semiconductor optic components. While each of the components listed above may not necessarily be required, they may be part of the particle therapy system. In addition to the one or more optical components, the particle therapy system may include non-optical components, such as electrical components, mechanical components, chemical reaction components, and semiconductor components. Such non-optical components may cooperate with optical components of the particle therapy system. For example, the optics components may include adaptive optics, such as an adaptive mirror connected to an actuator. In some embodiments, the electromagnetic radiation source may include one or more optics components to facilitate formation of the electromagnetic radiation beam. Additional details on optics components are described below with reference to FIGS. 1 and 2.

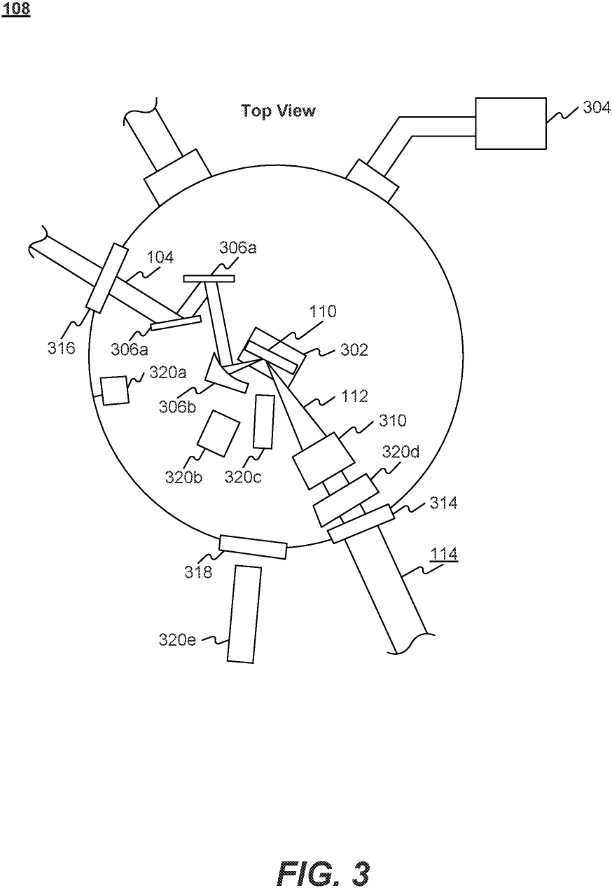

A particle therapy system in accordance with the present disclosure may include an interaction chamber configured to contain a target. As used in the present disclosure, the term “interaction chamber” may refer to any structure configured to isolate the target from ambient conditions and to provide an appropriate environment for particle generation. The interaction chamber may include one or more components for temperature adjustment, one or more components for pressure adjustment, one or more sensors for monitoring the conditions inside the interaction chamber, one or more sensors for monitoring properties associated with an interaction between an electromagnetic radiation beam and the target, one or more components for directing an electromagnetic radiation beam to the target, one or more particle beam adjustment components for directing charged particles, and more. Additional details on the interaction chamber are described below with reference to FIGS. 1 and 3.

A particle therapy system in accordance with the present disclosure may include a target for generating charged particles in response to electromagnetic irradiation. As used in the present disclosure, the term “target” may refer to any material, apparatus, or combination of elements configured for generating charged particles in response to energy application. As described below, the target may be configured for generating a proton beam; however, protons serve only as an example to the generated charged particles. In some embodiments, the target may be provided with a plurality of patterned features or microstructure elements. For example, the target may include a plurality of protrusions extending from a surface of the target. In some embodiments, the target may be patterned with one or more knife edges. For example, a knife edge of the target may include one or more narrow edges, similar to an arête or the edge of a blade. Consistent with the present disclosure, the particle therapy system may raster the electromagnetic radiation beam over the target. As used in the present disclosure, rastering may refer to a pattern of sequential scanning over a surface or volume having any shape. Rastering may be achieved by one or more motors configured to cause an electromagnetic radiation beam to sequentially scan a surface or volume. In some embodiments, an electromagnetic radiation beam may be rastered over individual patterned features of target, individual microstructure elements of target, or a knife edge of target. In some embodiments, an adaptive mirror may be configured to direct an electromagnetic radiation beam to strike individual features or individual microstructure elements of the target. Additional details on the target are described below with reference to FIGS. 1 and 3.

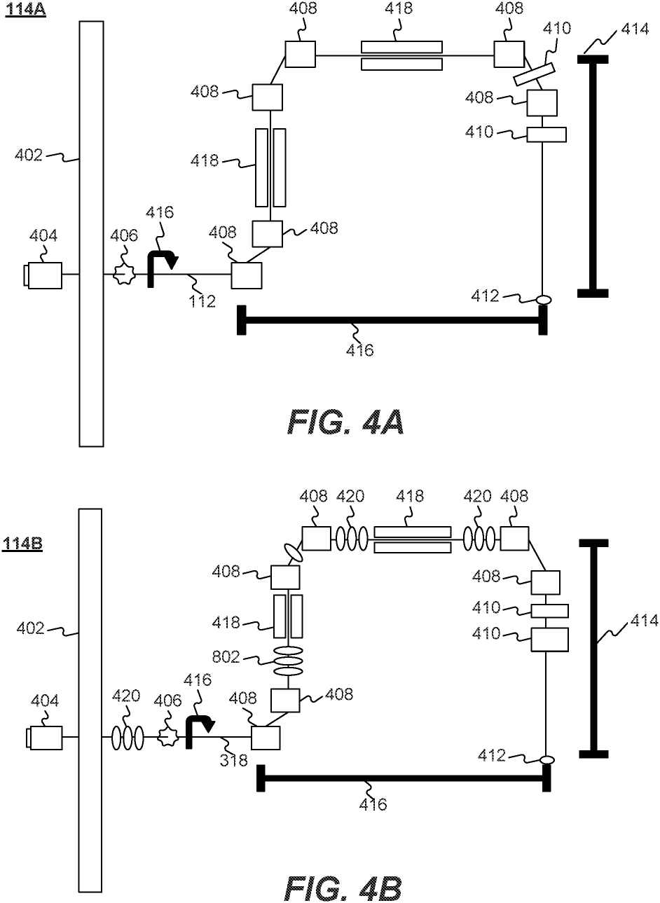

A particle therapy system in accordance with the present disclosure may include a beamline for delivering the generated charged particles to a patient. As used in the present disclosure, the term “beamline” may refer to any one or more components for manipulating and/or controlling a particle beam in any manner, including, for example, accelerating, analyzing, directing, shaping, filtering, splitting, delaying, modulating, absorbing, amplifying, focusing, chopping, and/or reflecting a particle beam. For example, a beamline may include one or more collimators, energy degraders, time-of-flight control units, magnetic dipoles, magnetic multipoles, solenoids or any other component suitable for manipulating charged particles. In some embodiments, the beamline may include a movable gantry for steering the beam of charged particles relative to the patient. A gantry may refer to any apparatus configured to assist in directing radiation toward a treatment volume. The treatment volume may include a group of cells or an area of tissue. For example, the treatment volume may be a tumor within a patient’s body. As mentioned above, a particle therapy system is merely one application of the disclosed systems; thus, the beamline may also be used to direct a particle beam toward different locations. Additional details on the beamline are described below with reference to FIGS. 4A and 4B.

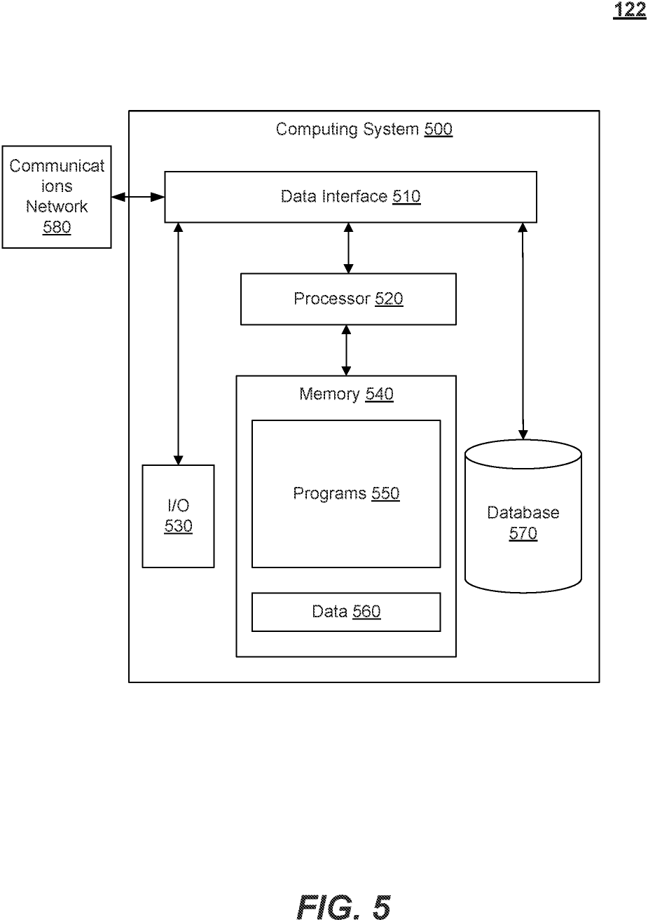

Consistent with disclosed embodiments, the particle therapy system may include or communicate with at least one processor configured to execute certain functions. The at least one processor may constitute any physical device having an electric circuit that performs a logic operation on an input or inputs. For example, the at least one processor may include one or more integrated circuits (ICs), including application-specific integrated circuits (ASICs), microchips, microcontrollers, microprocessors, all or part of a central processing unit (CPU), graphics processing unit (GPU), digital signal processor (DSP), field-programmable gate array (FPGA), or other circuits suitable for executing instructions or performing logic operations. Instructions executed by the at least one processor may be pre-loaded into a memory integrated with or embedded into the controller or may be stored in a separate memory. The memory may comprise a Random-Access Memory (RAM), a Read-Only Memory (ROM), a hard disk, an optical disk, a magnetic medium, a flash memory, other permanent, fixed, or volatile memory, or any other mechanism capable of storing such instructions. In some embodiments, the memory is configured to store information representative data about a scheduled treatment plan of a patient. In some embodiments, the at least one processor may include more than one processor. Each processor may have a similar construction, or the processors may be of differing constructions that are electrically connected or disconnected from each other. For example, the processors may be separate circuits or integrated in a single circuit. When more than one processor is used, the processors may be configured to operate independently or collaboratively. The processors may be coupled electrically, magnetically, optically, acoustically, mechanically or by other means that permit them to interact. Additional details on the processing unit and the at least one processor are described below with reference to FIG. 5.

### System Overview

FIG. 1 depicts an exemplary system 100 for providing particle therapy that includes an illustrative system for generating a beam of charged particles. In accordance with disclosed embodiments, particle therapy system 100 may include an electromagnetic radiation source 102 for generating an electromagnetic radiation beam 104, optics 106, an interaction chamber 108 including a target 110 for generating a particle beam 112, a beamline 114 for directing particle beam 112, a support platform 116 for supporting patient 118 with treatment volume 120, and a control system 122 configured to communicate with any one or more components of particle therapy system 100.

Consistent with the present disclosure, electromagnetic radiation source 102 may generate electromagnetic radiation beam 104 for applying energy to target 110. In some embodiments, electromagnetic radiation source 102 may comprise one or more gas lasers (e.g., CO₂ lasers), diode pumped solid state (DPSS) lasers (e.g., yitterbium lasers, neodymium-doped yttrium aluminum garnet lasers (Nd:YAG), or titanium-sapphire lasers (Ti:Sapphire)), and/or flashlamp-pumped solid-state lasers (e.g., Nd:YAG or neodymium glass). In a broader sense, any radiation source capable of causing a release of particles from target 110 may be employed. Electromagnetic radiation source 102 may be selected based on its intensity, i.e., the energy divided by the temporal duration of the pulse and the spot size of the laser on target 110. A variety of combinations of spatial profile (e.g., spot size, shape, and distribution), wavelength, temporal duration, and energy may be used while still providing the same intensity. For example, in some embodiments, electromagnetic radiation beam 104 may be within an energy range of 1 J to about 25,000 J, and a wavelength range of about 400 nm to about 10,000 nm. Electromagnetic radiation beam 104 may be pulsed, for example with a pulse width range of about 10 fs to 100 ns. Unless indicated otherwise, the term “about” with regards to a numeric value refers to a variance of up to approximately 5% with respect to the stated value. Electromagnetic radiation beam 104 may have various spot sizes. In some embodiments, a spot size between about 1 µm² and about 1 cm² may be used. Although spatial profiles of electromagnetic radiation beam 104 may have any beam profile, in some embodiments the spatial profile may include a Gaussian, super-Gaussian, Top Hat, Bessel, or annular beam profile.

As a more specific example, electromagnetic radiation source 102 may be a Ti:Sapphire laser. In the example of the Ti:Sapphire laser, electromagnetic radiation beam 104 may be within an energy range of about 1 J to 25 J and have a wavelength of about 800 nm. In this example, electromagnetic radiation beam 104 may have a pulse width range of about 10 fs to 400 fs, a spot size between about 2 µm² and 1 mm², and a Gaussian or Top Hat spatial profile. These properties are merely exemplary, and other configurations may be employed. Additional details on electromagnetic radiation source 102 are disclosed below with reference to FIG. 2.

Consistent with the present disclosure, electromagnetic radiation beam 104 may be directed to target 110 by one or more optics 106 disposed, for example, along a trajectory between electromagnetic radiation source 102 and target 110. Electromagnetic radiation beam 104 may include a defined energy, wavelength, power, energy, polarization (or it may not be polarized), spatial profile, and/or temporal profile. Any of the properties of electromagnetic radiation beam 104 may be fixed or may vary. Optics 106 may include one or more optical and/or mechanical components configured to alter properties of electromagnetic radiation beam 104, including spectral properties, spatial properties, temporal properties, energy, polarization, contrast ratio, or other properties. Optics 106 may include a wide variety of optical elements, such as lenses, mirrors, laser crystals and other lasing materials, piezo-activated mirrors, plates, prisms, beam splitters, filters, light pipes, windows, blanks, optical fibers, frequency shifters, optical amplifiers, gratings, pulse shapers, XPW, Mazzler (or Dazzler) filters, polarizers, Pockels cells, optical modulators, apertures, saturable absorbers, and other optical elements. Additional example and details on optics 106 are disclosed below with reference to FIG. 2.

Consistent with the present disclosure, electromagnetic radiation beam 104 may strike target 110 disposed within interaction chamber 108 configured to isolate target 110 from the outside environment. In a first example, interaction chamber 108 may contain a hydrogen-rich target, and the generated charged particles may be protons. In a second example, interaction chamber 108 may contain a carbon-rich target, and the generated charged particles may be carbon ions. In some embodiments, target 110 may have a surface on which microstructured elements are disposed. Such microstructured elements may be composed of one or more suitable materials, including ice (also referred to as “snow”), plastic, silicon, stainless steel, a variety of metals, carbon and/or any other material from which the particle beam may be generated. Such microstructured elements may be randomly arranged, arranged as defined by a growth or deposition process, and/or arranged in a patterned array. The microstructured elements may be configured based on one or more attributes of electromagnetic radiation beam 104. For example, such microstructured elements may have a dimension smaller than a wavelength of electromagnetic radiation beam 104, such less than third of the wavelength of electromagnetic radiation beam 104.

When target 110 is struck by electromagnetic radiation beam 104, it may emit a variety of particles, including electrons, protons, x-rays, and other particles. The emitted particles are used to form particle beam 112. Target 110 may be configured such that it includes one or more individual microstructured elements configured to interact with electromagnetic radiation beam 104. Alternatively or additionally, target 110 may include a continuous surface or texture formed from a material favorable for interaction with electromagnetic radiation beam 104. Those of skill in the art will understand that there are numerous configurations that may be employed to emit particles upon interaction with an electromagnetic radiation beam and that the disclosed details are merely exemplary.

Consistent with the present disclosure, beamline 114 is designed for forming a particle beam 112 from the particles emitted by target 110 and for directing particle beam 112 to treatment volume 120. Beamline 114 may be configured to be manipulated in one or more ways to influence the path of particle beam 112. Specifically, beamline 114 may include any equipment capable of manipulating charged particles, such as protons. For example, beamline 114 may include electromagnetic components. More specifically, beamline 114 may include one or more electromagnetic constituents, such as a quadrupole lens, cylindrical mirror lens/analyzer (CMA), spherical mirror lens/analyzer (SMA), collimator, energy degrader, time-of-flight control unit, magnetic dipole, or any other component suitable for manipulating charged particles. Beamline 114 may be used to adjust one or more properties of the particle beam 112. For example, beamline 114 may manipulate properties such as flux or spot size. Beamline 114 may filter particles having particular energies or reduce the energy of various particles. Beamline 114 may include one or more particle beam adjustment components disposed in various locations within particle therapy system 100. For example, the particle beam adjustment components may be located inside interaction chamber 108, within an associated beamline 114, or any combination thereof. In some embodiments, beamline 114 may be configured to maintain various conditions such as temperature, pressure (e.g., vacuum), or other conditions conducive to propagating and/or manipulating particle beam 112. Beamline 114 may further include other components for housing charged particle beams, including, but not limited to, elements such as beam dumps, beam attenuators, and protective shielding. Additional details of beamline 114 consistent with embodiments of the disclosure are discussed in further detail below with reference to FIGS. 4A and 4B.

Consistent with the present disclosure, patient 118 may be positioned on support platform 116. Support platform 116 may be any shape or form suitable for use with the other components of particle therapy system 100 and conducive to supporting patient 118 during treatment. Support platform 116 may be fixed in place relative to beamline 114, or support platform 116 may be configured for translation and/or rotation relative to beamline 114 prior to or during treatment. In some embodiments, support platform 116 may be adjusted to accommodate patients of different sizes or to position a treatment volume in a path of particle beam 112. In some embodiments support platform 116 may be adjusted during treatment to reposition the treatment volume relative to particle beam 112.

Control system 122 (or simply controller 122) may facilitate monitoring and/or control of various aspects of particle therapy system 100. For example, controller 122 may monitor various detectors associated with electromagnetic radiation source 102, optics 106, interaction chamber 108, beamline 114, and/or support platform 116. Controller 122 may also accept input from a user of system 100, such as a technician or other operator. Controller 122 may also accept, store, and/or execute operations pertaining to particle therapy system 100, including, for example, initiating and maintaining any functionalities of particle therapy system 100. In some embodiments, control system 122 may receive data associated with a treatment plan for treatment volume 120. Controller 122 may operate particle therapy system 100 to conform with the treatment plan. For example, controller 122 may define the energy levels of the particles that are to hit treatment volume 120. Controller 122 may be configured to implement feedback between one or more detectors and one or more of the various components of particle therapy system 100. For example, such feedback may improve precision, efficiency, speed, and/or other aspects of particle therapy system 100 or its operation. Examples of such feedback are described in greater detail below. Additional details of controller 122 are disclosed below with reference to FIG. 5.

### EM Source and Optics

FIG. 2 is a general schematic of electromagnetic radiation source 102. As shown in FIG. 2, electromagnetic radiation source 102 may include one or more oscillators 202, pump sources 204, optics components 206, diagnostics components 208, stretchers 210, amplifiers 212, compressors 214, and controllers 216. The configuration of FIG. 2 is merely an example, and numerous other configurations may be implemented consistent with the disclosed embodiments, incorporating one or more of the components of electromagnetic radiation source 102, system 100, or other components.

Oscillator 202 may include one or more lasers for generating an initial laser pulse 218 to be manipulated (e.g., shaped and/or amplified) to reach requirements for electromagnetic radiation beam 104. A wide variety of lasers or laser systems may be used as oscillator 202, including commercially available laser systems.

Pump source 204 may include independent lasers or laser systems configured to transfer energy into laser pulse 218. In some embodiments, pump source 204A may be connected to the output of oscillator 202 by an optical beamline incorporating one or more of optics 106. Additionally or alternatively, pump source 204 may include other pump mechanisms such as flash lamps, diode lasers, and diode-pumped solid-state (DPSS) lasers, or the like. In some embodiments, pump source 204 may be configured to alter a temporal profile of electromagnetic radiation beam 104. For example, control system 122 may be configured to control a timing of pump source 204A, thereby controlling characteristics of electromagnetic radiation beam 104, e.g., adjusting the timing of a pre-pulse of the electromagnetic radiation beam.

Optics components 206 may include any of the components discussed in relation to optics 106 and may perform any of the roles and/or functions described in relation to optics 106. Diagnostics 208 may include optical, opto-mechanical, and/or electronic components designed to monitor laser pulse 218, such as, its temporal and spatial properties, spectral properties, timing, and/or other properties. More specifically, diagnostics 208 may include one or more photodiodes, oscilloscopes, cameras, spectrometers, phase sensors, auto-correlators, cross-correlators, power meters or energy meters, laser position and/or direction sensors (e.g., pointing sensors), dazzlers or mazzlers, etc. Diagnostics 208 may also include or incorporate any of the components identified above with respect to optics components 206.

Stretcher 210 may be configured to chirp or stretch laser pulse 218. More specifically, stretcher 210 may include diffraction gratings or other dispersive components, such as prisms, chirped mirrors, and the like. Amplifier 212 may comprise an amplification medium such as, for example, titanium sapphire crystal, CO₂ gas, or Nd:YAG crystal. Amplifier 212 may include a holder for the amplification medium. The holder may be configured to be compatible with supporting environmental conditions, such as positioning, temperature, and others. Amplifier 212 may be configured to receive energy from additional pump source 204B and transfer this energy to laser pulse 218.

Compressor 214 may include one or more optical components configured to compress laser pulse 218 temporally, for example to a final temporal duration. Compressor 214 may be constructed from diffraction gratings positioned on holders and positioned in a vacuum chamber. Alternatively, compressor 214 may be constructed of dispersion fibers or prisms. Compressor 214 may include mirrors or other optics components 306, motors, and/or electronically controlled opto-mechanics.

Controller 216 may include one or more electronic systems that control and/or synchronize various components of electromagnetic radiation source 102. Controller 216 may include any combination of controllers, power supplies, computers, processors, pulse generators, high-voltage power supplies, and/or other components. As an example, controller 216 may include one or more computing systems 500, which may be dedicated to electromagnetic radiation source 102 or shared with other components of system 100. In some embodiments, some or all of the functions of controller 216 may be performed by controller 122 of system 100.

Controller 216 may interface with various components of electromagnetic radiation source 102 and other components of system 100 via communication channels. The communication channels may be configured to transmit electrical or other signals to control various optical or opto-mechanical components associated with electromagnetic radiation source 102 or system 100. The communication channels may include a conductor compatible with high voltage, electrical triggers, various wired or wireless communication protocols, optical communications, or other components.

Consistent with the present disclosure, electromagnetic radiation beam 104 may be manipulated by optics 106. For example, optics 106 may be configured for various uses, such as laser beam steering, laser beam diagnostics, laser-target interaction diagnostics, and/or target viewing and positioning. Optics 106 may be disposed in various places along the path of electromagnetic radiation beam 104 between electromagnetic radiation source 102 and target 110, or in any other system of system 100 where optical components are desired. In some embodiments, optics 106 may be tailored to parameters related to an intended beam. For example, optics 106 may be tailored in terms of wavelength, intensity, temporal pulse shape (e.g., pulse width), spatial size and energy distribution, polarization, and other properties of the intended beam. Such beam parameters may relate to an optics substrate material, size (e.g., lateral size or thickness), coating material (if any), shape (e.g., planar, spherical, or other), orientation relative to a beam, or other specifications.

Optics 106 may be disposed in specific environmental conditions, such as a vacuum and/or an environment purged by one or more gasses. In some embodiments, the lifespan of optics 106 may vary. Some optics 106 may be long-term equipment, reused numerous times. Alternatively or additionally, some optics 106 may be consumable, used fewer times and replaced. Such classification may be based on a number of factors such as laser intensity and presence of debris/contamination. In some use cases, debris shielding may be installed proximate to expensive or delicate optics to reduce a need for frequent replacements. Periodic examination may be performed for optics suspected to be damaged. Specialized optical systems may be installed to examine optics at risk. In addition, optics 106 may be manipulated manually, automatically, or by any combination thereof. Input types for manipulating optics 106 may include high voltage signals, triggering signals, optical pumping, or any other form of input. Further, optics 106 may be monitored by one or more cameras, such as CCD cameras. Automatic manipulation of adaptive mirrors may occur, for example, in response to one or more signals provided by the control system 122. The control system 122 may control one or more motors, piezoelectric elements, microelectromechanical (MEMS) elements, and/or the like associated with a deformable mirror. Alternatively or additionally, the control system 122 may control one or more laser pulses, anti-reflective coated substrates, and/or the like associated with a plasma mirror.

Consistent with the present disclosure, optics 106 may be fixed or adaptive. For example, optics 106 may include one or more active, adaptive, or reconfigurable components, such as deformable mirrors, plasma mirrors, Pockels cells, phase shifters, optical modulators, irises, shutters (manually or computer controlled), and other similar components. In some embodiments, the adaptive properties of optics 106 as well as properties of electromagnetic radiation beam 104 may be manipulated, as in the case of a deformable mirror or plasma mirror. Examples of deformable mirrors that may be included in optics 106 include, for example, segmented mirrors, continuous faceplate mirrors, magnetic mirrors, MEMS mirrors, membrane mirrors, bimorph mirrors, and/or ferrofluidic mirrors. Any number of other mirror technologies capable of altering the wave front of electromagnetic radiation beam 104 may also be used. Examples of plasma mirrors that may be employed in optics 106 include a laser pulse focused onto an anti-reflective coated substrate. The plasma mirrors may be established by directing the laser pulse towards a parabolic mirror located in front of the anti-reflective coated substrate, or any other way.

In some implementations, optics 106 may include one or more adaptive optics. As used in the present disclosure, an adaptive optics may refer to an element that includes a reflective surface that may be adapted. For example, an adaptive mirror may be a deformable mirror that comprises a plurality of facets, each of the plurality of facets being independently controllable by digital logic. As another example, an adaptive mirror may be a plasma mirror that comprises a laser pulse focused onto an anti-reflective coated substrate, one or both of the laser pulse and anti-reflective coated substrate being controllable by digital logic. In some embodiments, an adaptive mirror may be configured to direct electromagnetic radiation beam 104 at target 110, thereby facilitating formation of particle beam 112. An adaptive mirror in accordance with the present disclosure may be configured to adjust or control a spatial profile of electromagnetic radiation beam 104 and/or to adjust or control at least one of a relative position and orientation between electromagnetic radiation beam 104 and target 110. In some instances, an adaptive mirror may be configured to direct electromagnetic radiation beam 104 by adjusting one or more property of electromagnetic radiation beam 104. For example, adjustment may be achieved by at least one of adjusting a focus of electromagnetic radiation beam 104, diverting electromagnetic radiation beam 104, and scanning electromagnetic radiation beam 104.

In some embodiments, the adaptive mirror may be configured such that electromagnetic radiation beam 104 will sequentially or simultaneously strike a plurality of locations on target 110 or a plurality of targets 110 disposed in different locations within system 100. In such configurations, an adaptive mirror or other optics 106 may alter the path of electromagnetic radiation beam 104 to direct the beam onto the multiple locations and/or plurality of targets. For example, an adaptive mirror or other optics 106 may sequentially divert (e.g., scan) electromagnetic radiation beam 104 from one location to an adjacent location in a pattern continuously or intermittently, such as in a stepwise manner. In an automated process, control system 122 may be configured to cause the adaptive mirror to direct electromagnetic radiation beam 104 at predetermined locations on the surface of target 110. For example, it may be advantageous to scan electromagnetic radiation beam 104 over a patterned array of particle-generating features or microstructure elements provided at a surface of target 110. It may also be advantageous to scan electromagnetic radiation beam 104 over target 110 that includes a plurality of particle-generating structures substantially oriented along a common axis, such as protrusions substantially extending away from a surface of target 110. It may also be advantageous to scan electromagnetic radiation beam 104 over target 110 patterned with one or more knife edges, such as target that includes one or more features having a narrow edge similar to an arête or the edge of a blade. The adaptive mirror is described as an example. Those of skill in the art will recognize that other optics 106 may perform the same or similar functions as those described above in reference to the adaptive mirror.

### Interaction Chamber and Target

FIG. 3 depicts an example of an interaction chamber 108. Interaction chamber 108 may be any size and shape; and may be constructed of any appropriate material or materials capable of housing a target during laser-target interaction and for isolating the target from the outside environment. Stainless steel is one example of a material that may be used to construct the interaction chamber 108. In some embodiments, the interaction chamber may include a target stage. As used in the present disclosure, a target stage may refer to any structure configured to support target. In some embodiments, a target stage may be controlled by a processor configured to cause relative movement between the target stage and an electromagnetic radiation beam. For example, interaction chamber 108 may include one or more stages 302 configured to support target 110 and/or other equipment within interaction chamber 108, such as optics components, beam adjustment components, detectors, or the like.

The orientation of stage 302 may be fixed or adjustable by causing a translation and/or rotation along one or more axes. In one embodiment, stage 302 may be associated with a motor and the movement of the motor may adjust stage 302 to alter the relative orientation between electromagnetic radiation beam 104 and target 110. Specifically, stage 302 may be associated with one or more corresponding holders configured to hold stage 302 in place while allowing positioning of stage 302 to an appropriate degree of accuracy, for example translation and rotation, as well as other degrees of freedom. Such degrees of freedom may be manipulated manually or via any appropriate automatic means, such as electric motors. The adjustment of stage 302 may be manual or automated. Automated adjustment may be performed, for example, in response to one or more signals provided by control system 122. For example, feedback signals may relate to measured properties of the laser-target interaction, and, in response, control system 122 may provide a control signal to a motor connected to stage 302.

In some embodiments, stage 302 may optionally be configured to heat, cool, or maintain the temperature of target 110. The temperature control may be achieved, for example, by monitoring the temperature of target 110 and raising, lowering, or maintaining the temperature of target 110 in response to the measured temperature. In one example, temperature monitoring may be achieved with one or more thermocouples, one or more infrared temperature sensors, and/or any other technique used to measure temperature. Temperature adjustment may be made, for example, by adjusting the amount of electric current flowing through a heating element. The heating element may be, for example, a refractory metal such as tungsten, rhenium, tantalum, molybdenum niobium, and/or alloys thereof. Temperature adjustment may also be made, for example, by flowing a coolant, such as water or a cryogenic fluid (e.g., liquid oxygen, liquid helium, liquid nitrogen, etc.) through a conduit directly or indirectly placed in thermal communication with target 110. As a person of ordinary skill in the art would appreciate, these exemplary manners of adjusting temperature are compatible and may be combined. Of course, these temperature adjustment methods are not limiting, and any other known method for heating, cooling, and or maintaining the temperature of target 110 may be used with the disclosures herein.

Consistent with the present disclosure, upon striking target 110, an interaction of electromagnetic radiation beam 104 and target 110 may generate various charged particles that may be used in particle beam 112. In some embodiments, charged particles may be emitted at a particle energy of about 250 MeV from a location on target 110 struck by electromagnetic radiation beam 104 focused to a spot size of about 10 to 100 µm. The two-dimensional divergence angle of charged particles emitted from target 110 may be about 0.2 radians (i.e., about 11 degrees). In addition, particle energy angular distribution ∂Ω/∂E and particle number energy distribution ∂N/∂E may be very small so that the energy angular distribution and particle number energy distribution are reasonably constant. As an example, a pulse of electromagnetic radiation beam 104 may result in the emission of about 10⁸ charged particles, and pulses may be repeated at a rate of about 10 to 1000 Hz. Accordingly, a pulsed electromagnetic radiation beam 104 may thereby produce a pulsed particle beam 112. A pulse of charged particles may also be referred to as a particle “bunch.”

Interaction chamber 108 may also include one or more vacuum pumps 304. For example, either or both of sample preparation and particle beam formation may have sub-atmospheric pressure requirements or may achieve optimal performance within a particular range of sub-atmospheric pressures. Vacuum pump 304 may be used to influence pressure conditions within interaction chamber 108 and/or components associated with interaction chamber 108. For example, vacuum pump 304 may maintain a vacuum condition or near-vacuum condition in interaction chamber 108. Examples of vacuum pump 304 may include one or more turbo-molecular pumps, cryogenic pumps, particle pumps, or mechanical pumps, such as diaphragm or roots pumps. Vacuum pump 304 may operate in conjunction with one or more pressure regulators and/or valves (not shown in the figures).

Interaction chamber 108 may also include optics components 306. Any of the components noted above with respect to optics 106 may be used inside the interaction chamber to direct electromagnetic radiation beam 104. For example, as shown in FIG. 3, interaction chamber may include mirrors 306a configured to direct electromagnetic radiation beam 104 toward target 110. In addition, interaction chamber 108 may include a parabolic mirror 306b configured to focus electromagnetic radiation beam 104 onto target 110. Interaction chamber 108 may also include any number of particle beam adjustment components associated with beamline 114. For example, interaction chamber 108 may include a collimator 310. Those of skill in the art will appreciate that alternatively or additionally, other particle beam adjustment components may be incorporated into interaction chamber 108 to facilitate propagation the charged particles towards beamline 114.

Interaction chamber 108 may also include one or more valves 314. Any suitable valve may be used and may be located, for example, between various portions of interaction chamber 108 or between interaction chamber 108 and other components of system 100 or its ambient environment. Valve 314 may be configured, for example, to isolate vacuum pump 304 or beamline 114. Valve 314 may be manual or automatic. Automatic valves may be, for example, pneumatic and/or electronic. Valve 314 may be simple open/close valves, such as a two-position gate valve, or valve 314 may be configured to be partially open. Valve 314, associated with vacuum pump 304, may include one or more butterfly valves that can vary continuously between open and closed states. Valve 314 may be configured to maintain pressure, retain or release materials, and/or allow access to interaction chamber 108 for maintenance of parts or replacement of targets.

Interaction chamber 108 may also include one or more shutters 316. Shutter 316 may be configured to block or allow electromagnetic radiation beam 104 into interaction chamber 108. In some examples, shutter 316 may be a simple open/close shutter. Shutters 316 may also be configured to chop electromagnetic radiation beam 104 if desired. Operation of shutter 316 may be manual or automated. Automated operation may occur, for example, in response to one or more signals provided by control system 122. Interaction chamber 108 may also include one or more windows 318. Windows 318 may be composed of any material suitable for the pressure, temperature, and other environmental factors associated with interaction chamber 108.

Interaction chamber 108 may also include one or more sensors 320. Sensors 320 may be configured to measure conditions associated with interaction chamber 108. As used herein, a sensor may refer to a device that detects one or more properties of a sample chamber condition, an electromagnetic radiation source or beam, a particle beam, and/or a laser-target interaction. Sensor 320 may observe any condition within and/or proximate to the interaction chamber. In some embodiments, a system for generating a particle beam may include other sensors separate from an interaction chamber. As an example, a sensor may be configured to measure at least one laser-target interaction property. As used in the present disclosure, a laser-target interaction may refer to an observable property related to the interaction of an electromagnetic radiation beam with target. Laser-target interaction properties may include, for example, a particle beam property, a secondary electron emission property, an x-ray emission property, a particle beam energy, a particle beam flux, and/or other property indicative of the interaction between electromagnetic radiation beam 104 and target 110. In some embodiments, measurements may be taken on a single-shot basis. That is, sensors 320 may be configured to measure properties associated with an individual interaction between electromagnetic radiation beam 104 and target 110. Sensors 320 may also measure the same or different properties on a more continuous basis, for example, providing results after processing. The placement of sensors 320 may vary based on a number of factors, including space constraints and optimal location for measurement. As shown in FIG. 3, sensors 320 may be located along an outer wall of interaction chamber 108 (such as sensor 320a), proximate to target 110 (such as sensors 320b and 320c), or in line with particle beam 112 (such as 320d).

For some sensors 320, there may be an advantage to detection proximate to target 110, and thus to interaction between electromagnetic radiation beam 104 and target 110 (laser-target interaction). In an embodiment, system 100 may be stabilized over time, after which such proximity may be unnecessary. In some embodiments, one or more sensors 320 may be mounted outside of interaction chamber 108. For example, FIG. 3 depicts sensor 320e outside interaction chamber 108 proximate to window 318. Sensors 320 may be disposed such that they are inherently subject to properties intended to be measured or conditions within interaction chamber 108 and may be altered to facilitate measurement. For example, optics 106 may include a steering mirror configured to temporarily, intermittently, or continuously deflect a signal from an interaction area to a sensor outside interaction chamber 108. For example, sensor 320e may receive signal through window 318. The above detector placements are merely exemplary, and numerous others may be apparent to those of skill in the art.

In some embodiments, one or more sensors 320 may be configured to measure one or more laser-target interaction properties of electromagnetic radiation beam 104 or particle beam 112. In some embodiments, sensors 320 may include quadrupole analyzers, spherical mirror analyzers (SMAs), cylindrical mirror analyzers (CMAs), secondary electron detectors, photomultipliers, scintillators, solid-state detectors, time-of-flight detectors, laser-on-target optical diagnostic detectors, x-ray detectors, cameras, Faraday cups, or other detectors. Sensors 320 may detect properties such as absorption or reflection, a secondary electron emission property, a plasma property such as electron temperature and/or density, and/or an x-ray emission property. Secondary emissions, such as emission of electrons and/or x-rays may be indicative of laser-target interaction properties and/or properties of particle beam 112. For example, the energy spectrum and/or flux of electrons and/or x-rays may indicate particle beam properties. These signals may then be used as input in a feedback loop for modifying the laser-target interaction, for example, by adjusting one or more of electromagnetic radiation source 102, one or more optics components, one or more particle beam adjustment components, and the position/orientation of target 110, as described in greater detail below.

Consistent with the present disclosure, sensors 320 may detect particle beam direction, spatial spread, intensity, flux, energy, particle energy, and/or energy spread. For example, in some embodiments, a Thompson parabola may be employed. In such embodiments, particle beam 112 may be directed into an area in which magnetic and electric fields deflect the charged particles to locations on a detection screen. The location at which the charged particles contact the screen may indicate particle energy. For such a screen, any particle-sensitive device may be used, such as CR-39 plates, image plates, and/or scintillators (coupled to an imaging device such as a CCD camera). As another example, spatial particle beam distribution may be detected with a screen sensitive to charged particles, such as CR-39 and image plate or a scintillator with a detection device (such as a camera).

Sensors 320 may also include a time-of-flight detector. The time-of-flight detector may measure average particle energy. In some embodiments, the time-of-flight detector may include a particle scintillator and a detector with adequate temporal resolution, such as a photo-multiplier-tube (PMT). The time when the particle signature is detected on the PMT may indicate particle velocity and thus particle energy. Sensors 320 may also include instruments configured for plasma diagnostics, such as x-ray spectrometers configured to detect electron temperature and density, or interferometers configured to detect plasma density. Optical diagnostics may include imaging of the reflected laser beam to measure the laser absorption efficiency. These detectors may be used during initial system design, calibration, and testing, and they may optionally be included in the final system.

In some embodiments, target 110 may be prefabricated. In other embodiments, target 110 may be produced in situ within system 100 or an attached sample preparation system. For example, target 110 may disposed within an interaction chamber, such as interaction chamber 108. This may involve forming target from a suitable material, including forming such material on a substrate. Such materials may include any gas, solid, or liquid chemical sources of the types commonly known in techniques such as evaporation, physical vapor deposition, chemical vapor deposition, molecular beam epitaxy, atomic layer deposition, and the like. For example, in embodiments in which target 110 includes ice, materials used to form the targets may include water vapor (H₂O), hydrogen gas (H₂), and/or oxygen gas (O₂). In embodiments in which target 110 includes silicon, materials used to form target 110 may include, for example, silane (SiH₄), disilane (Si₂H₆), trichlorosilane (SiHCl₃), or any other silicon source. In embodiments in which target 110 includes plastic, sources may include, for example, polytetrafluoroethylene (PTFE) polymer source materials or any other PTFE source. As a person of ordinary skill in the art would recognize, these are just a few illustrative examples among many available target materials and target source materials. In addition, the interaction chamber may vary in structure to suit the form of the target employed. For example, when the target is ice, the interaction chamber may be specifically configured to maintain an appropriate temperature to support the ice. Each target material may have differing sustaining requirements, and therefore the structure of interaction chamber 108 may vary to suit the type of target 110.

In related embodiments, system 100 may also include a separate or substantially separate preparation chamber connected to interaction chamber 108 and configured for target preparation and/or conditioning. The preparation chamber may include various equipment and instrumentation for preparing targets, such as equipment that may be found in systems for performing evaporation, physical vapor deposition, chemical vapor deposition, molecular beam epitaxy, atomic layer deposition, and the like. The preparation chamber may also include temperature control elements, one or more sample transfer mechanisms, such as a transfer arm or any transfer device known by those familiar with vacuum systems. Additionally, system 100 may also include a load lock between the preparation chamber and interaction chamber 108.

### Beamline

Interaction chamber 108 may include or interface with a particle beamline 114, as illustrated in FIGS. 4A and 4B. The beamline may include a plurality of components for particle beam adjustment and reconfiguration for appropriately directing particle beam 112 prior to and during a treatment. For example, beamline 114A and beamline 114B may include a solenoid 404, a coupling 406, beam adjustment components 408, collimators 418, scanning magnets 410, and/or other components. In some embodiments, beamline 114 may include a conduit maintained at sub-atmospheric pressures to facilitate propagation of beam of charged particles. Accordingly, beamline 114A and beamline 114B may also include vacuum pumps (not shown) to achieve and/or maintain sub-atmospheric conditions.

Consistent with the present disclosure, controlling a relative movement between a particle beam and a treatment volume in two dimensions of a three-dimensional coordinate system for delivering particle beam 112 to an isocenter 412 may be achieved in numerous ways. For example, controlling the relative movement between particle beam 112 and treatment volume 120 may be achieved by rotating a gantry which may include beamline 114. Alternatively or additionally, controlling the relative movement between particle beam 112 and treatment volume 120 may be achieved by directing a particle beam with an electromagnet and/or moving support platform 116. In some embodiments, isocenter 412 may represent the location of treatment volume 120 or a location within treatment volume 120. A height 414 and a length 416 of beamline 114 may vary based on numerous possible configurations of beamline 114. In some embodiments either or both of height 414 and length 416 may be as little as 2 meters. In addition, beamline 114 may be separated from other components of system 100 by a wall 402 or other barriers. Wall 402 may include one or more openings (not shown) to allow passage of particle beam 112 and any beamline or other equipment configured to deliver particle beam 112. Location of wall 402 may vary based on a number of factors, and in some embodiments wall 402 may not be present.

As illustrated in FIG. 4A, beamline 114A may include solenoid 404. Solenoid 404 may be configured to capture charged particles (e.g., protons) emitted by target 110. In some embodiments, charged particles emitted by target 110 may exhibit a large divergence. As an example, beam size of charged particles emitted from target 110 may expand by a factor of 100 over a short distance, such as 1 cm. Solenoid 404 may be configured to reduce convergence of particle beam 112. In some embodiments, solenoid 404 may include a high-field solenoid, such as, for example, a superconducting solenoid at 9 to 15 T. Field strength may be related to solenoid length and resulting beam size. Higher solenoid field strength may result in smaller beam size and aperture required in solenoid 404. Solenoid 404 may vary in length based on field strength and other factors. In some embodiments, solenoid 404 may be between 0.55 m and 0.85 m in length with an aperture between 4 cm and 20 cm. In some embodiments, solenoid 404 may be used in conjunction with one or more collimators, and in some embodiments the charged particles may be captured by a series of quadrupoles. As one example, at least three quadrupoles may be used to correct the strong initial divergence. Specifically, a solution can be obtained with normal conducting quadrupoles or with superconducting quadrupoles.

Beamline 114A may include coupling 406. Coupling 406 may be any mechanical and or optical connection configured to facilitate physical movement of beamline 114, such as rotation about an axis of rotation. Beamline 114 may be configured to be physically moved by any appropriate arrangement of actuators, which may be controlled by controller 122. Coupling 406 may include one or more bearings or bushings and may be connected to and/or integrated into beamline 114. oupling 406 may be configured to maintain a seal or other barrier to prevent loss of a vacuum state or other environmental conditions within beamline 114.Coupling 406 may include rotationally invariant optics, for example to reduce tune dependence as a function of beamline position.

Beamline 114A may include one or more beam adjustment components 408. Beam adjustment components 408 may include any of beam adjustment components discussed above, configured to guide particle beam 112 through the beamline. In some embodiments, beam adjustment components 408 may include electromagnets, such as dipoles and/or quadrupoles. Beam adjustment components 408 may include normal conducting dipoles, superferric dipoles, superconducting coil dipoles, stripline dipoles, etc. In some embodiments, beam adjustment components 408 may include dipole pairs (e.g., each bending particle beam 112 by approximately 45°) to form a rectangle or any other combinations of angles to form a rectangle or another desired shape. The dipole pairs may operate at about 4.8 T and be about 0.6 m long. Straight sections between dipole pairs may be adjusted independently, providing tuning range flexibility and customization of the electromagnetic optics. Splitting 90° bends into two may improve reference trajectory control, as each dipole may be adjusted independently, such as via shunts on a single power supply, providing at least 10% variation (20% total relative change for two bends). Thus, the dipole pairs may facilitate independent trajectory correction on each arm of beamline 114, increasing tolerances and reducing cost.

Beamline 114A may include one or more collimators 418. Collimators 418 may be configured to filter particle beam 112 such that only charged particles traveling in a desired direction and/or having a desired momentum are allowed to pass. Collimators 418 may be disposed in a variety of locations within beamline 114. For example, if beam adjustment components 408 have achromatic properties producing undesired effects on the beam downstream, collimators 418 may be configured to counteract such effects.

Beamline 114A may include one or more scanning magnet 410. Scanning magnets 410 may include beam adjustment components configured to adjust the location in space of isocenter 412. Scanning magnets 410 may be controlled by control system 122, such as to adjust location of treatment being provided to treatment volume 120. Scanning magnets 410 may be disposed in any of a number of locations within beamline 114. For example, scanning magnets 410 may be upstream from one or more of beam adjustment components 408, downstream of all of beam adjustment components 408, or a combination of such upstream and downstream locations, as shown in FIG. 4A.

System 100 may be configured such that scanning magnets are operated in cooperation with other components to control the location of treatment within patient 118. For example, control system 122 may control any combination of scanning magnets 410, movement of beamline 114, and/or movement of patient support platform 311. One or more components may be configured for control of particular dimensions and/or degrees of freedom. For example, patient support platform 116 may be configured to adjust patient position in one dimension, while scanning magnets 410 adjust in a dimension orthogonal to the first. Alternatively or additionally, system 100 may be configured such that a coarse adjustment in a given dimension may be performed by a different component than a fine adjustment. For example, a coarse adjustment in a particular dimension may be performed by a motor configured to manipulate patient support platform 116, while fine adjustment may be performed by a scanning magnet 410. Numerous combinations of such adjustments will be apparent to those of skill in the art.

FIG. 4B depicts a further example of beamline 114. Beamline 114B may include some or all of the same components as beamline 114A, such as solenoid 404, coupling 406, beam adjustment components 408, collimators 418, and scanning magnets 410, and may further include additional quadrupole elements 420. Quadrupole elements 420 are magnetic elements that are part of the magnetic beamline and help deliver particle beam 112 to treatment volume 120 with the desired transmission and size. Quadrupole elements 420 are typically used to focus or de-focus a beam of charged particles. Quadrupole elements 420 may be permanent magnets (e.g., made of rear-earth elements and/or other magnetic materials), normal-conducting electromagnets, super-conducting coil electromagnets, pulsed magnets, or other devices capable of providing the appropriate fixed or tunable magnetic field.

In particle therapy, particles of certain energies are needed to irradiate a treatment volume located at a particular depth within a patient. To isolate charged particles of the desired energies, beamline 114 may be designed for filtering some of the particles of particle beam 112 to deliver the particles having the desired energies to the patient. For example, to deliver charged particles having energies in a desired energy level, beamline 114 may filter a particle bunch by removing any charged particles having energies less than a first energy threshold and particles having energies greater than a second energy threshold. In some embodiments, such filtering may be achieved by combining certain particle beam adjustment components. For example, beamline 114 may manipulate particle beam 112 such that charged particles having certain energies are diverted along a different trajectory than charged particles having other energies. This may be achieved in a number of ways. For example, beamline 114 may include a band-pass filter to isolate charged particles having the desired energy level. In another embodiment, beamline 114 may include a high-pass filter to isolate charged particles having energies greater than an energy cut-off. In another embodiment, beamline 114 may include a low-pass filter to isolate charged particles having energies less than an energy cut-off.

The above embodiments may be combined, and more than one filter may be used. A low-pass filter and a high-pass filter may be combined in series, for example, to create a band-pass filter. In such an embodiment, the low-pass filter may be configured to isolate charged particles having energies less than the first energy threshold, and the high-pass filter may be configured to isolate charged particles having energies greater than the second energy threshold. This may be particularly advantageous for selecting charged particles within a narrow energy band, especially an energy band narrower than a stand-alone band-pass filter can accommodate.

### Control System

FIG. 5 is a diagram of an exemplary controller 122 consistent with disclosed embodiments. As a person of ordinary skill will understand, some or all of the functions associated with controller 122 may be executed or facilitated by software, hardware, or any combination thereof associated with a computing system 500. In one embodiment, computing system 500 may have one or more data interfaces 510, one or more processors 520, one or more memories 540, and one or more input/output (I/O) devices 530. In some embodiments, computing system 500 may take the form of a server, general purpose computer, customized dedicated computer, mainframe computer, laptop, mobile device, or any combination of these components. In certain embodiments, computing system 500 (or a system including computing system 500) may be configured as a particular apparatus, system, or the like based on the storage, execution, and/or implementation of software instructions that may perform one or more operations consistent with the disclosed embodiments. Computing system 500 may be standalone, or it may be part of a subsystem, which may be part of a larger system.

One or more data interfaces 510 may be used to communicate directly or indirectly with a plurality of software-driven components system 100. For example, data interface 510 may communicate with I/O devices 530 and database 570. The term “data interface” may include any device or system configured to receive digital data from one or more sources. The disclosed embodiments are not limited to any particular data interface configurations or protocol. The specific design and implementation of data interfaces 510 may depend on the communication networks over which computing system 500 is intended to operate. For example, in some embodiments, computing system 500 may include a data interface 510 designed to operate and receive digital data from communications network 580, e.g., over the Internet, a Local Area Network, a cellular network, a public switched telephone network (PSTN), or other suitable communications network. Consistent with embodiments of the present disclosure, data interfaces 510 may receive data associated with a treatment plan for a tumor of patient 118.

At least one processor 520 may include one or more known processing devices, such as an application specific integrated circuit (ASIC), a digital signal processor (DSP), a programmable logic device (PLD), a field programmable gate array (FPGA), a processor, a controller, a microprocessor, other electronic units, or combination thereof. At least one processor 520 may constitute a single core or multiple core processor that executes parallel processes simultaneously. For example, at least one processor 520 may be a single core processor configured with virtual processing technologies. In certain embodiments, at least one processor 520 may use logical processors to simultaneously execute and control multiple processes. At least one processor 520 may implement virtual machine technologies, or other known technologies to provide the ability to execute, control, run, manipulate, store, etc. multiple software processes, applications, programs, etc. In other embodiments, at least one processor 520 may include a multiple-core processor arrangement (e.g., dual, quad core, etc.) configured to provide parallel processing functionalities to allow computing system 500 to execute multiple processes simultaneously. One of ordinary skill in the art would understand that other types of processor arrangements could be implemented that provide for the capabilities disclosed herein. The disclosed embodiments are not limited to any number or type of processors.

Memory 540 may include one or more storage devices configured to store instructions used by at least one processor 520 to perform functions related to the disclosed embodiments. For example, memory 540 may be configured with one or more software instructions, such as programs 550 that may perform one or more operations when executed by at least one processor 520. The disclosed embodiments are not limited to separate programs or computers configured to perform dedicated tasks. For example, memory 540 may include a program 550 that performs the functions of computing system 500, or program 550 could comprise multiple programs. Additionally, at least one processor 520 may execute one or more programs located remotely from computing system 500. For example, controller 122, may, via computing system 500 (or variants thereof), access one or more remote programs that, when executed, perform functions related to certain disclosed embodiments. At least one processor 520 may further execute one or more programs located in a database 570. In some embodiments, programs 550 may be stored in an external storage device, such as a server located outside of computing system 500, and processor 520 may execute programs 550 remotely.

Memory 540 may store data that may reflect any type of information in any format that computing system 500 may use to perform operations consistent with the disclosed embodiments. For example, memory 540 may store instructions to enable at least one processor 520 to execute one or more applications, such as server applications, network communication processes, and any other type of application or software. Alternatively, the instructions, application programs, etc., may be stored in an external storage (not shown) in communication with computing system 500 via a suitable network, including a local area network or the internet. Memory 540 may be a volatile or non-volatile, magnetic, semiconductor, tape, optical, removable, non-removable, or other type of storage device or tangible (i.e., non-transitory) computer-readable medium. Memory 540 may include data 560. Data 560 may include any form of data used by controller 122 in controlling particle therapy treatment via system 100. For example, data 560 may include data related to operation of various components of system 100, feedback parameters associated with various components of system 100, data gathered from one or more detectors associated with system 100, treatment plans for particular patients or for particular diseases, calibration data for various components of system 100, etc.

I/O devices 530 may include one or more devices configured to allow data to be received and/or transmitted by computing system 500. I/O devices 530 may include one or more digital and/or analog communication devices that allow computing system 500 to communicate with other machines and devices, such as other components of system 100 shown in FIG. 1. For example, computing system 500 may include interface components that may provide interfaces to one or more input devices, such as one or more keyboards, mouse devices, displays, touch sensors, card readers, biometric readers, cameras, scanners, microphones, wireless communications devices, or the like that may enable computing system 500 to receive input from an operator of controller 122. I/O devices may include one or more devices configured to allow controller 122 to communicate with one or more of the various devices of system 300, such as through wired or wireless communication channels.

Computing system 500 may contain one or more databases 570. Alternatively, computing system 500 may be communicatively connected to one or more databases 570. For example, computing system 500 may be communicatively connected to database 570 via a communications network, such as a wired or wireless network. Database 570 may include one or more memory devices that store information and are accessed and/or managed through computing system 500. In some embodiments, database 570 is stored at a location separated from computing system 500, and the data from database 570 is obtained using communications network 580 and data interface 510.

Communications network 580 facilitates communications and sharing of data between computing system 500 and other databases. Communications network 580 may be any type of network that provides communications, exchanges information, and/or facilitates the exchange of information between communications system 580 and other devices. For example, communications network 580 may be the Internet, a Local Area Network, a cellular network, a public switched telephone network (PSTN), or other suitable connection(s) that enables system 100 to send and receive data relevant for treating patient 118.

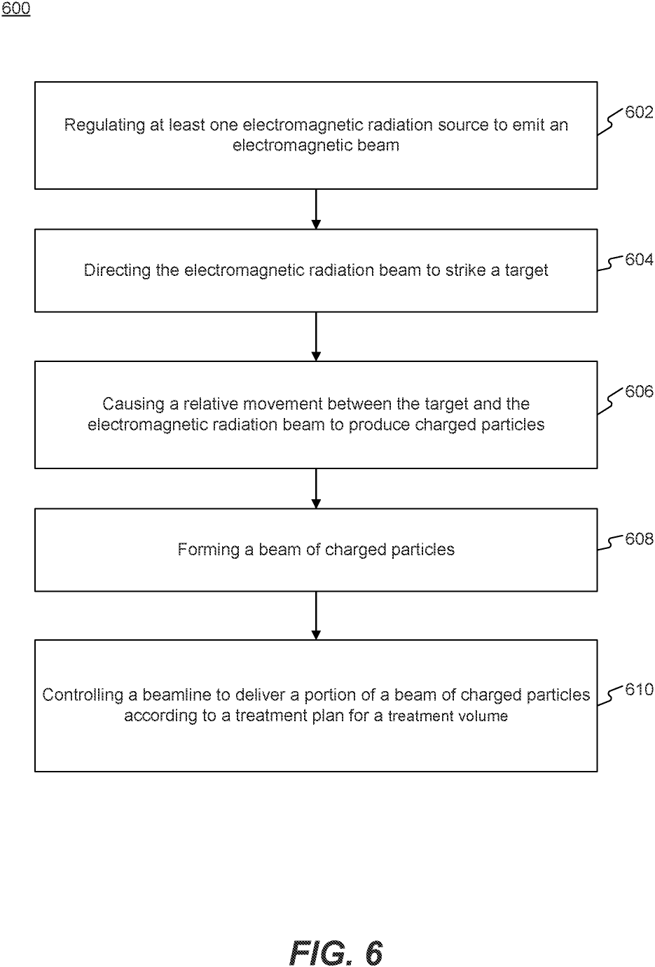

FIG. 6 is an exemplary flowchart depicting a process 600 executable by a processing device (e.g., at least one processor 520) for treating patient 118 with charged particles. The steps of process 600 may be carried out automatically, such as by control system 122. The steps of process 600 may be carried out in response to user input, such as through control system 122 or carried out by a combination of automatic and manual operation of various components. In some embodiments, process 600 may be carried out based on specifications in a treatment plan, which may be customized to varying degrees based on a particular patient, treatment type, and/or treatment volume.

In step 602, the processing device may regulate at least one electromagnetic radiation source (e.g., electromagnetic radiation source 102) to emit an electromagnetic beam an electromagnetic radiation source (e.g., electromagnetic radiation beam 104). In step 604, the processing device may direct the electromagnetic radiation beam to strike a target (e.g., target 110). Electromagnetic radiation beam 104 may be generated and directed via any components capable of radiation beam generation, such as, for example, various combinations of the components described in relation to FIG. 2.

In step 606, the processing device may cause a relative movement between the target and the electromagnetic radiation beam to produce charged particles. In some embodiments, the surface of target 110 may be scanned by electromagnetic radiation beam 104. For example, electromagnetic radiation beam 104 may be sequentially scanned over the surface of target 110 by continuous or intermittent rastering, stepwise scanning, or any other scanning waveform desired. Alternatively, electromagnetic radiation beam 104 may be non-sequentially scanned over the surface of target 110. Electromagnetic radiation beam scanning may be achieved by manually or automatically adjusting optics 106 located between electromagnetic radiation source 102 and target 110. Automatic adjustment of optics 106 may be achieved, for example, in response to one or more signals provided by control system 122. The one or more control signals provided by control system 122 may be predetermined by a program, such as a program stored in computing system 500, and they may be provided in response to one or more feedback signals received from various elements of system 100, such as one or more sensors. For example, information from the one or more sensors in system 100 may indicate that altering the location of the laser-target interaction site is desirable.

In step 608, the processing device may use particle beam adjustment components to form a particle beam (e.g., particle beam 112) from the charged particles. The charged particles generated in step 608 initially may not be disposed in a useful configuration or trajectory. The charged particles may be formed into a particle beam, for example, by one or more beam adjustment components of beamline 114. Properties of the particle beam may vary based on the configuration of system 100 and from use to use. In one embodiment, the particle energies may be about 250 MeV, as noted above, and may range, for example, from 60 to 250 MeV. The particle flux may be about 2 Gy/min, and particle pulse duration may be less than about 100 psec. The charged particles generated by system 100 may also have a symmetric phase space profile, allowing improvements in particle beam steering and filtering over accelerator-based particle generation systems, thereby improving the accuracy and the efficiency of particle beam delivery and treatments. Of course, the above ranges are only examples, and the specific energies and flux may vary based on particulars of the configuration.

In step 610, the processing device may control a beamline (e.g., beamline 114) to deliver a portion of a beam of charged particles (e.g., particle beam 112) according to a treatment plan for a treatment volume (e.g., treatment volume 120). In some embodiments, the beam of charged particles may pass through a zone proximate to a particle beam adjustment component being part of beamline 114. The zone may be of any size, but in some embodiments may have a dimension of less than one inch. The zone proximate to particle beam adjustment component may be configured and/or oriented for a particle beam (e.g., a continuous beam or a pulsed beam including particle pulses) to traverse the zone. The particle beam adjustment component may include any of particle beam adjustment components, for example, an electromagnet such as a dipole, CMA, SMA, or time-of-flight analyzer. As the particle beam traverses the zone proximate to particle beam adjustment component, an automated switch may activate particle beam adjustment component such that charged particles having the desired energy range are diverted along a first trajectory and charged particles having energies outside the desired range are diverted along a second trajectory, for example, toward a beam dump.

### A Particle Therapy System With Electromagnetic Sources Having High Repetition Rate

Performances of a particle therapy system consistent with the present disclosure (e.g., system 100) depend on the characteristics of its electromagnetic radiation source. Specifically, a particle therapy system with a laser that emits pulses at a high repetition rate can generate more charged particles to treat a patient than a particle therapy system with a laser that emits pulses at a lower repetition rate. However, simply upgrading the laser in the particle therapy system to a high repetition laser by itself will not yield optimal performances. The particle therapy system itself needs to be designed to utilize the electromagnetic radiation source to its maximum potential. The following disclosure describes a particle therapy system having a high power and high repetition electromagnetic radiation source.

In disclosed embodiments, a particle therapy system may include an electromagnetic radiation source (e.g., electromagnetic radiation source 102) characterized by a plurality of working parameters (e.g., power, repetition rate, wavelength, pulse duration, coherence length, polarization, and more). The electromagnetic radiation source may generate a pulsed electromagnetic radiation beam (e.g., electromagnetic radiation beam 104). In one example, the generated pulsed electromagnetic radiation beam may be characterized by a power of at least 100 terawatts, for example, at least 150 terawatts, at least 250 terawatts, or at least 500 terawatts. The power of electromagnetic radiation source 102 may describe the average power of a pulsed laser and may be measured in watts (W). Pulsed lasers are also characterized by their pulse energy, which is proportional to average power and inversely proportional to the electromagnetic radiation source’s repetition rate. The pulse energy may be measured in joules (J). The generated pulsed electromagnetic radiation beam may be characterized by a repetition rate of at least 20 Hz, for example, at least 30 Hz, at least 50 Hz, or at least 100 Hz. The repetition rate of electromagnetic radiation source 102 (also known as pulse repetition frequency) may describe the number of pulses emitted every second. As mentioned above, the repetition rate is inversely proportional to pulse energy and directly proportional to average power. Higher repetition rates may result in less thermal relaxation time at the surfaces of the optics and at the final focused spot, which leads to more rapid material heating.

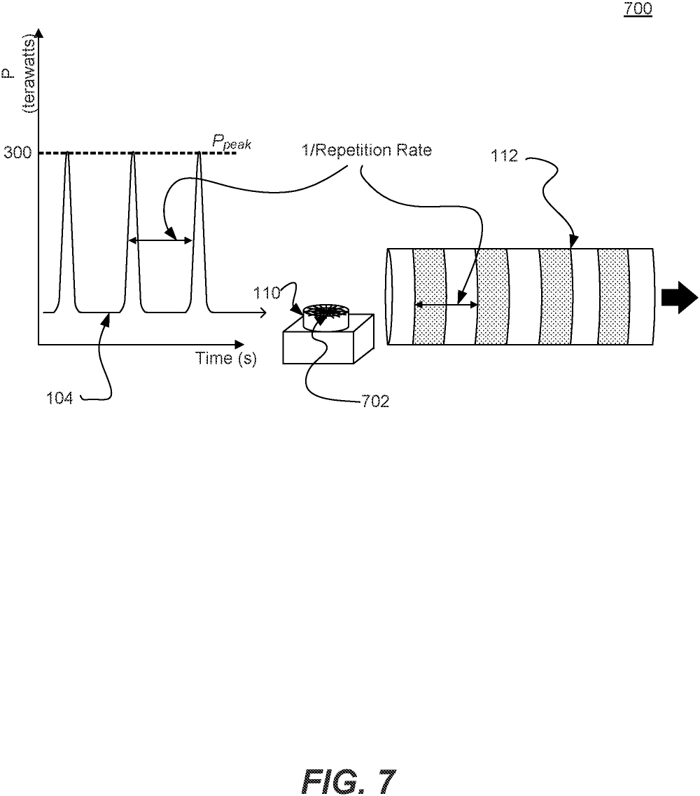

FIG. 7 depicts an example illustration 700 of a pulsed electromagnetic radiation beam 104 hitting target 110 and generating pulsed particle beam 112. In the illustrated example, the pulse peak power (Pₚₑₐₖ) may be 300 terawatts, and the repetition rate may be 20 Hz. Thus, the 1/repetition rate may be 0.05 sec. In other implementations, however, the pulse peak power may be greater than 300 terawatts and the repetition rate may be greater than 20 Hz. For example, the repetition rate may be up to 100 Hz. Particle therapy system 100 may also include optics 106 (not shown in FIG. 7) configured to direct pulsed electromagnetic radiation beam 104 along a path towards target 110 that may be located in interaction chamber 108 (not shown in FIG. 7). The result of the interaction of pulsed electromagnetic radiation beam 104 on a surface of target 110 may cause a resultant pulsed emission from target 110 of at least about 3×10⁶ charged particles per pulse. In some embodiments, the resultant emission includes negatively charged particles (e.g., electrons) for delivery to patient 118. In alternative embodiments, the resultant emission includes positively charged particles (e.g., protons or ions) for delivery to patient 118. Specifically, interaction chamber 108 may contain a hydrogen-rich target such that the charged particles included in particle beam 112 may be protons. Alternatively, interaction chamber 108 may contain a carbon-rich target and the charged particles included in particle beam 112 may be carbon ions. A person skilled in the art would recognize that other electromagnetic radiation sources characterized by different set of working parameters may generate a different pulsed electromagnetic radiation beam 104 that may also cause a resultant pulsed emission from target 110 of at least about 3×10⁶ charged particles per pulse.

Consistent with the present disclosure, target 110 may be sized to enable at least a plurality of locations of interaction 702 with pulsed electromagnetic radiation beam 104. For example, target 110 may be sized to enable at least 50 locations of interaction, at least 100 locations of interaction, at least 175 locations of interaction, at least 350 locations of interaction, or at least 500 locations of interaction with pulsed electromagnetic radiation beam 104. The term “location of interaction” in this disclosure refers to a point or region on the surface of the target with which the pulsed electromagnetic radiation interacts. Typically, the size of the location of interaction depends on the spot size of pulsed electromagnetic radiation beam 104 and associated with the beam diameter at the focal point of optics 106. In some particle therapy systems, a desired goal may be minimizing the spot size to maximize power density. To do so, one or more aspheric lenses may be used instead of conventional spherical lenses to reduce spherical aberrations and decrease the focal spot size. In some configurations, every time pulsed electromagnetic radiation beam 104 hits the surface of target 110, a crater is formed. The diameter of such a crater depends on the power of electromagnetic radiation source 102. For example, an average diameter of a crater when a 100 terawatts laser is being used can be between about 1 µm and 10 mm. The phrase “a target sized to enable X locations of interaction with pulsed electromagnetic radiation beam” may mean, for example, that the area of the target is greater than about X times the average area of the craters formed at the target. In one embodiment, control system 122 may use an estimated average area of a crater to determine the locations of interaction in target 110. Preferably, the locations of interaction in target 110 should be used only one time for particle generation. In some cases partially overlapping locations of interaction in target 110 may be used as long as the overlapping part is less than a predefined value, for example, less than 25%, less than 15%, or less than 10%.

In some embodiments, the surface of target 110 may include a plurality of microstructured features, and electromagnetic radiation source 102 may destroy some of the microstructured features at each location of interaction between electromagnetic radiation source 102 and target 110. The terms “microstructured features” and “microstructured elements” refer to features of a surface that have at least one dimension (e.g., height, length, width, or diameter) of less than one millimeter. The microstructured features may be purposely imposed on the surface of target 110 and do not include inadvertent formations on the microstructured material. Imposition of the microstructured features on the surface of target 110 may include forming the microstructured features by modifying a surface of an existing layer to generate the microstructured features and/or depositing material onto a surface already having microstructured features. The microstructured features can be formed to have at least one dimension of no more than about 100 micrometers, about 10 micrometers, or less. The term “microstructured layer” refers to a layer having a surface that includes microstructured features. Moreover, each location of interaction may include one or more microstructured element. The term “microstructured element” refers to an individual microstructured feature of the surface of target 110 that extends away from the surface of target 110 and is separate from other microstructured elements. A plurality of microstructured features disposed on a surface of target 110 may have the same cross-section shape (e.g., circle, ellipse, etc.) or different shapes. The plurality of microstructured features may be of the same size or of different sizes. In particular, the size of each microstructured element may vary in a random, pseudorandom, or a planned manner. In one example, the size of each microstructured element may be associated with a height and with a width. The height of each microstructured element may be measured normal to the surface of target 110, and the width of each microstructured element may be measured in the plane of the surface of target 110. In the context of the disclosure, the term “width” refers to the maximum dimension of a microstructured element in the plane of the surface of target 110. For example, when the microstructured element is a circle, the width would be the diameter of the circle, and when the microstructured element is an ellipse, the width would be the major diameter of the ellipse.

In one embodiment, the height distribution (and/or the width distribution) of the plurality of microstructured features may follow a probability density function. The probability density function may include Normal Distribution, Uniform Distribution, Cauchy Distribution, Chi-Square Distribution, Gamma Distribution, Beta Distribution, or others. For example, the height of the plurality of microstructured features may be distributed normally about an average size of about 10 µm. In one embodiment, the average height of the plurality of microstructured features may be greater than about 1 µm and less than about 1 mm. In accordance with another embodiment, an average width of the plurality of microstructured features may be greater than about 100 nm and less than about 100 µm. For example, the average width of the plurality of microstructured features may between about 50 nm and 50 µm. Additionally, the plurality of microstructures may be part of a target with a fractal-like morphology, which offers a wide range of self-similar features with sizes ranging from tens of nanometers to a few microns in the focal volume of the electromagnetic radiation pulses. For example, amorphous snow targets may have a fractal-like morphology.

Particle therapy system 100 may further include an actuator configured to cause relative movement between target 110 and electromagnetic radiation beam 104 at a speed associated with the repetition rate of electromagnetic radiation source 102 to thereby vary a location of interaction of pulsed electromagnetic radiation beam 104 on a surface of target 110. This enables causing a resultant emission from target 110 of at least about 3×10⁶ charged particles per pulse. In other words, to ensure emission of enough charged particles per pulse, particle therapy system 100 may confirm that electromagnetic radiation beam 104 does not hit the same location of interaction twice (or at least strikes partially overlapping locations of interaction). Consistent with the present disclosure, the speed of the relative movement between target 110 and electromagnetic radiation beam 104 may be associated with a rate equal to or exceeding the repetition rate of electromagnetic radiation source 102.

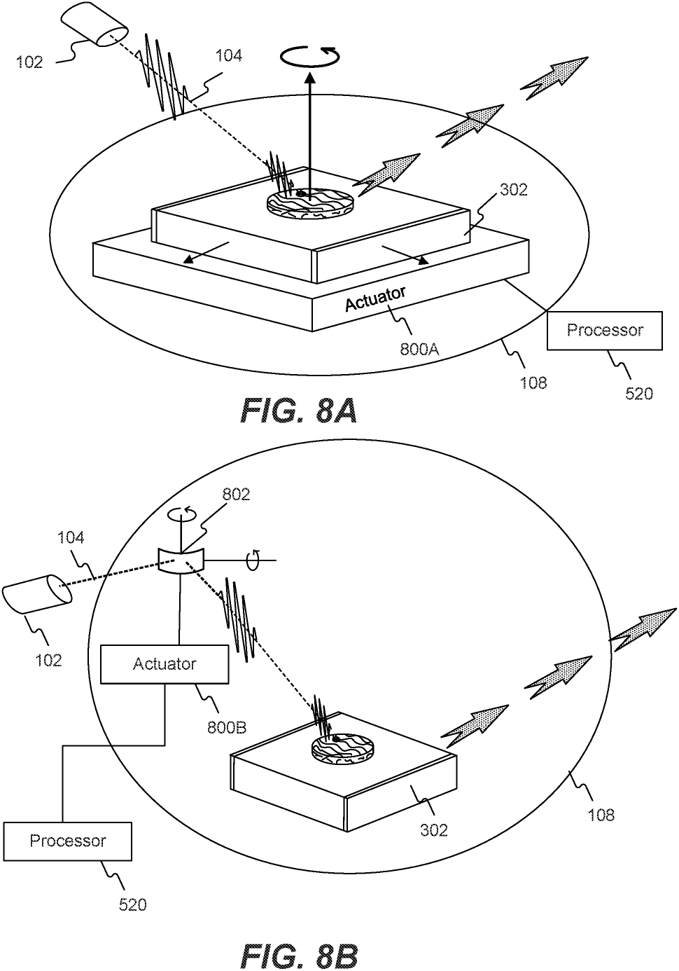

FIGS. 8A and 8B are diagrammatic illustrations of at least one actuator 800 configured to cause relative movement between target 110 and electromagnetic radiation beam 104. FIG. 8A illustrates a first actuator 800A configured to cause movement of target 110 within interaction chamber 108 and FIG. 8B illustrates a second actuator 800B configured to cause a change in the path of electromagnetic radiation beam 104.

In FIG. 8A, stage 302 is placed on top of first actuator 800A and may be configured to alter the relative orientation between electromagnetic radiation beam 104 and target 110. Specifically, actuator 800A is configured to cause movement of target 110 within interaction chamber 108 by moving stage 302. As shown, actuator 800A is configured to cause translation movement and/or rotation movement of target 110. In one embodiment, actuator 800A may be configured to linearly move target stage 302 by at least 20 mm/s. For example, actuator 800A may be configured to linearly move target stage 302 by at least 30 mm/s, at least 50 mm/s, or by at least 750 mm/s. Additionally or alternatively, actuator 800A may be configured to rotate stage 302 or rotate target 110 at a speed of at least 0.5 RPM. For example, actuator 800A may be configured to rotate stage 302 or rotate target 110 at a speed of at least 0.75 RPM, at least 1.5 RPM, or at least 3 RPM. In some cases, the movement of target 110 caused by actuator 800A may be continuous or non-continuous (i.e., having small jumps).

In FIG. 8B, the optics of system 100 may include an adjustable mirror 802, and actuator 800B is configured to alter the relative orientation between electromagnetic radiation beam 104 and target 110. Specifically, actuator 800B may vary the orientation of adjustable mirror 802 located within interaction chamber 108 to cause a change in the path of electromagnetic radiation beam 104. In one embodiment, adjustable mirror 802 may be a single axis square deformable mirror, and actuator 800B may be made of semiconductor (e.g., silicon) and may include a piezoelectric layer (e.g., PZT, lead zirconate titanate, aluminum nitride) that changes its dimension in response to electric signals applied by an actuation controller (e.g., processor 520), a semiconductive layer, and a base layer. The physical properties of actuator 800B may determine the mechanical stresses that actuator 800B experiences when electric current passes through it. When the piezoelectric material is activated, it exerts force on actuator 800B and causes it to bend, which may cause adjustable mirror 802 to change the path of electromagnetic radiation beam 104. In another embodiment, adjustable mirror 802 may be a dual-axis round deformable mirror, and actuator 800B may include four actuators. Consistent with some embodiments, a dual-axis mirror may be configured to deflect electromagnetic radiation beam 104 in a horizontal direction and in a vertical direction. For example, the angles of deflection of a dual-axis mirror may be between about 0° to 30° in the vertical direction and between about 0° to 30° in the horizontal direction. One skilled in the art will appreciate that adjustable mirror 802 may have numerous variations and modifications. In some cases, the changes in the path of electromagnetic radiation beam 104 caused by actuator 800B may be continuous or non-continuous (i.e., having small jumps).

Consistent with the present disclosure, control system 122 may control at least one actuator such that the relative movement between target 110 and the electromagnetic radiation beam 104 would correlate with the working parameters of electromagnetic radiation source 102 (e.g., power, repetition rate, pulse duration, etc.) to avoid hitting the same location of interaction on the target more than a predetermined number of times or to avoid hitting at least partially overlapping locations of interaction more than a predetermined number of times. For example, electromagnetic radiation beam 104 may avoid hitting the same location of interaction on the target more than once, more than twice, more than three times, etc. In a first embodiment, at least one processor 520 may control actuator 800A such that the movement of target 110 would correlate with a pulsed electromagnetic radiation beam of at least 100 terawatts and at a repetition rate of at least 20 Hz to avoid hitting the same location of interaction on the target more than a predetermined number of times or to avoid hitting at least partially overlapping locations of interaction more than a predetermined number of times. In a second embodiment, processor 520 may control actuator 800B such that the change in the path of electromagnetic radiation beam 104 is determined based on knowing that the power of electromagnetic radiation beam 104 is at least 100 terawatts and its repetition rate is at least 20 Hz to avoid hitting the same location of interaction on the target more than a predetermined number of times or to avoid hitting at least partially overlapping locations of interaction more than a predetermined number of times.

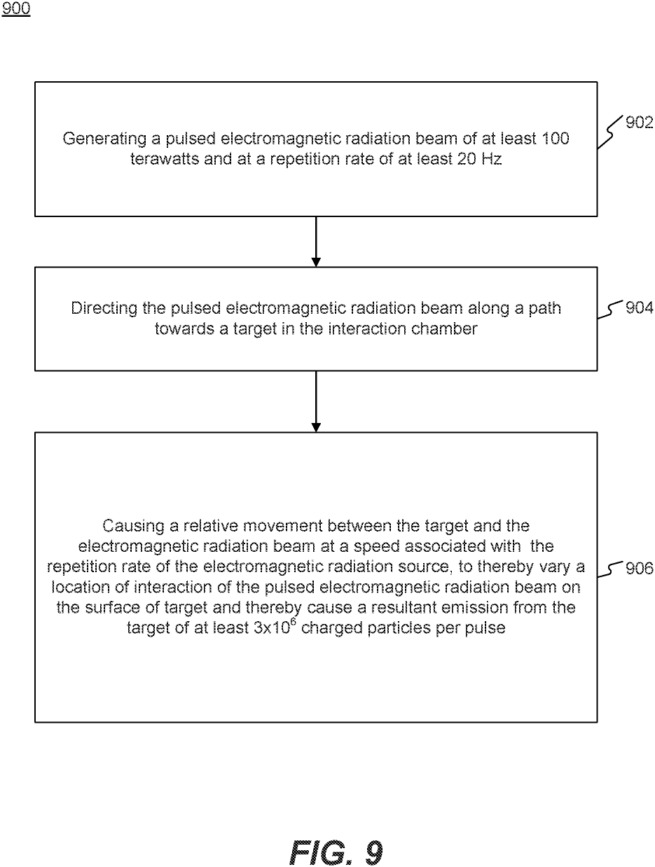

FIG. 9 is a flowchart of an example process 900 for particle therapy executed by a processing device of control system 122 according to embodiments of the present disclosure. For purposes of illustration, in the following description reference is made to certain components of particle therapy system 100. It will be appreciated, however, that other implementations are possible and that any combination of components or devices may be utilized to implement the exemplary method. It will also be readily appreciated that the illustrated method can be altered to modify the order of steps, delete steps, or further include additional steps, such as steps directed to optional embodiments.

Disclosed embodiments may include “generating a pulsed electromagnetic radiation beam of at least 100 terawatts and at a repetition rate of at least 20 Hz.” As discussed earlier, pulsed electromagnetic radiation beam 104 may be generated by electromagnetic radiation source 102 that may be characterized by a plurality of working parameters. By way of example only, according to step 902 in FIG. 9, the processing device may generate a pulsed electromagnetic radiation beam of at least 100 terawatts and at a repetition rate of at least 20 Hz. In a specific example, the processing device may activate electromagnetic radiation source 102 having a power of 115 terawatts and repetition rate of 30 Hz. The activation of electromagnetic radiation source 102 by the processing device may be initiated after control system 122 completed a series of tests to confirm that the generated particle beam 112 is going to hit treatment volume 120. The series of tests includes confirming that target 110 is located where it should be, that patient 118 is located where he or she should be, and more.

Disclosed embodiments may include “directing the pulsed electromagnetic radiation beam along a path towards a target in the interaction chamber.” As discussed earlier, pulsed electromagnetic radiation beam 104 may be directed using optics 106 that may include adaptive optics configured to adjust or control a spatial profile of electromagnetic radiation beam 104 and/or to adjust or control at least one of a relative position and orientation between electromagnetic radiation beam 104 and target 110. By way of example only, according to step 904 in FIG. 9, the processing device may direct pulsed electromagnetic radiation beam 104 along a path towards target 110. The processing device may direct pulsed electromagnetic radiation beam 104 by controlling adaptive optics (e.g., controlling an adjustable mirror to change the path of the electromagnetic radiation beam). In some embodiments, target 110 may be sized to enable at least 100 locations of interaction with pulsed electromagnetic radiation beam 104. The processing device may selectively direct pulsed electromagnetic radiation beam 104 to non-overlapping locations on target 110. For example, non-overlapping locations on target 110 may be locations with less than about 5% overlapping in the area of the crater caused when electromagnetic radiation beam 104 hits the surface of target 110. Alternatively, the processing device may selectively direct pulsed electromagnetic radiation beam 104 to partially overlapping locations on target 110. For example, partially overlapping locations on target 110 may be locations with between about 5% and about 50% overlapping in the area of the crater caused when electromagnetic radiation beam 104 hits the surface of target 110. In some embodiments, the surface of target 110 may include a plurality of microstructured elements, and each location of interaction may include at least one microstructured element. As explained in greater detail below, electromagnetic radiation source 102 may be configured to destroy differing microstructured elements at each differing location of interaction.

Disclosed embodiments may include “causing a relative movement between the target and the electromagnetic radiation beam at a speed associated with the repetition rate of the electromagnetic radiation source, to thereby vary a location of interaction of the pulsed electromagnetic radiation beam on the surface of target and thereby cause a resultant emission from the target of at least 3×10⁶ charged particles per pulse.” As discussed earlier, the relative movement between target 110 and electromagnetic radiation beam 104 may be caused by moving target 110 (e.g., using first actuator 800A), by changing the path of electromagnetic radiation beam 104 (e.g., using second actuator 800B), or by any combination thereof. Consistent with the present disclosure, the relative movement includes movement of target 110 within interaction chamber 108 and/or controlling an adjustable mirror within interaction chamber 108. In some cases, the processing device may rotate target 110 at a speed of at least 0.5 RPM and/or linearly move target 110 by at least 20 mm/s. By way of example only, according to step 906 in FIG. 9, the processing device may cause a relative movement between target 110 and electromagnetic radiation beam 104 at a speed associated with the repetition rate of the electromagnetic radiation source 102. In one embodiment, the speed may be equal to or exceed the repetition rate of electromagnetic radiation source 102. The processing device causes the relative movement to vary a location of interaction of pulsed electromagnetic radiation beam 104 on the surface of target 110 and thereby causes a resultant emission from target 110 of at least about 3×10⁶ charged particles per pulse. The charged particles may be negatively charged particles or positively charged particles. In a first example, interaction chamber 108 may contain a hydrogen-rich target and the generated charged particles may be protons. In a second example, interaction chamber 108 may contain a carbon-rich target and the generated charged particles may be carbon ions.

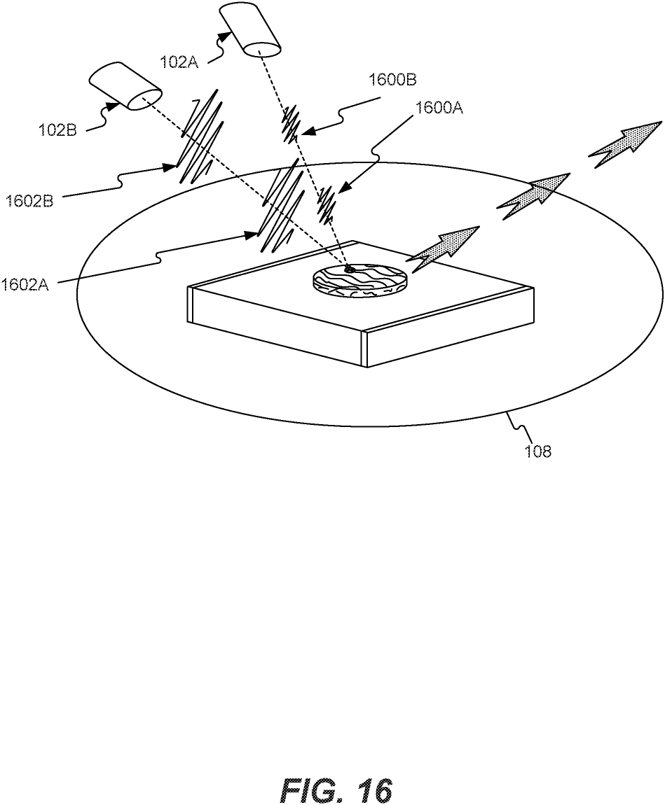

In one embodiment, a pulsed electromagnetic radiation beam may include a plurality of pulse chains, each pulse chain including a preliminary pulse and a main pulse. The preliminary pulse may exceed an energy flux threshold and have an energy flux on the target of between about 0.1 and 10 J/cm². The main pulse may have an intensity on the target of at least about 10¹⁸ W/cm². A time separation between the preliminary pulse and the main pulse may be between about 1 ns and 26 ns, such that during the time separation the target is free from irradiation exceeding the energy flux threshold. This embodiment is discussed in greater detail with reference to FIGS. 16 through 18 below.

### A Particle Therapy System That Initiates Changes to Target Location as Particle Beam Direction Changes

Operating a particle therapy system as described in the present disclosure involves control over different parts and subsystems. For example, various faults may occur in relation to the electromagnetic radiation source, the optics, the target located in the interaction chamber, or the beamline that directs the emitted charged particles. To render a successful treatment session, the operation of all parts and subsystems of the particle therapy system are coordinated. As used in this specification, the term “successful treatment session” refers to delivering at least about 95% of the charged particles specified in the treatment plan to the treatment volume at the desired energy level. The following disclosure explains how the particle therapy system may coordinate operation of its different parts and subsystems during a treatment session.

In disclosed embodiments, a particle therapy system may include an interaction chamber (e.g., interaction chamber 108) configured to contain a target (e.g., target 110) that emits charged particles in response to energy application. The system may include an energy source (e.g., a laser, such as electromagnetic radiation source 102) for applying energy to the target. The particle therapy system may include a magnetic beamline (e.g., beamline 114) for directing a beam of charged particles (e.g., particle beam 112) from the target to a tumor of a patient (e.g., patient 118) in a manner that enables charged particles to strike the tumor at differing tumor locations. The particle therapy system may include at least one processor (e.g., control system 122) configured to selectively direct energy from the energy source to differing locations on the target. During a treatment session, when locations of energy application on the target may change, the at least one processor may selectively control a relative movement between the beam of charged particles and the patient to strike the tumor with charged particles at differing tumor locations.

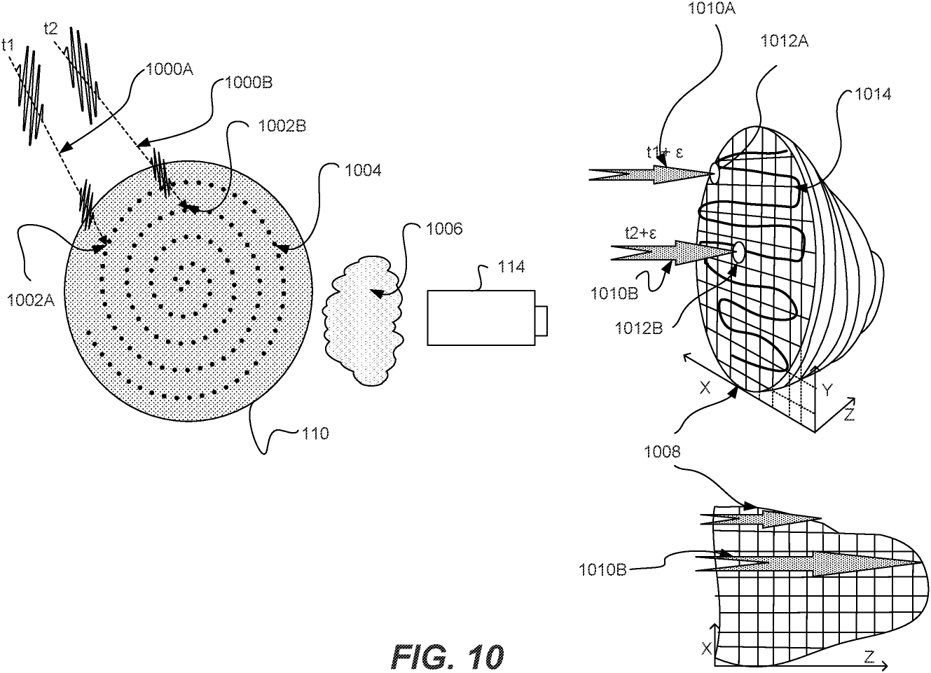

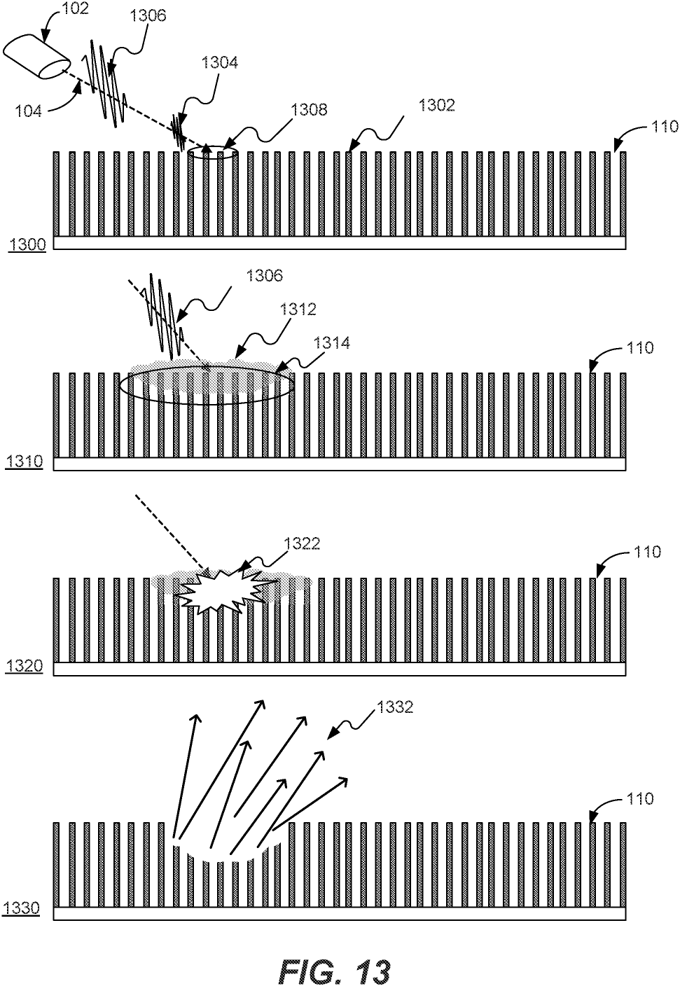

FIG. 10 depicts an example of the particle therapy process. A first pulse 1000A of electromagnetic radiation beam 104 interacts with a surface of target 110 at a first location of interaction 1002A at first time t1. Thereafter, at second time t2, a second pulse 1000B of electromagnetic radiation beam 104 interacts with a surface of target 110 at a second location of interaction 1002B. A third pulse may interact with a surface of target 110 at a third location of interaction and a third time t3, and so on. Consistent with the present disclosure, target 110 may contain a plurality of microstructured elements thereon, and electromagnetic radiation beam 104 may destroy one or more microstructured elements at each of the differing locations of interaction 1002 on target 110. Additional details on this embodiment are described below with reference to FIG. 13. As described above, control system 122 may cause the location of interaction 1002 at the surface of target 110 to change such that electromagnetic radiation beam 104 does not hit the exact same spot twice. In some embodiments, control system 122 may selectively direct energy from the energy source to non-overlapping locations on target 110. In other embodiments, control system 122 may selectively direct energy from the energy source to partially overlapping locations on target 110, for example, less than 25% overlap, less than 15% overlap, or less than 5% overlap. Spiral path 1004 illustrates an example of possible locations of interaction during a treatment session. In some cases, spiral path 1004 may be denser (i.e., the locations of interaction may be closer to each other) or less dense based on the treatment plan, the treatment type, treatment volume, and/or the estimated duration of the treatment session. As used in this specification, the term “treatment session” refers to a medical procedure during which a patient is treated with charged particles. The procedure may be performed within a certain period, such as within a day, several hours, several minutes, or other duration of time. In some embodiments, a patient may remain in a treatment room and/or on a patient support (e.g., support platform 116) during the treatment session. In some embodiments, control system 122 may selectively direct energy from the energy source to a plurality of differing locations on target 110 during a single treatment session. The plurality of differing locations on target 110 during a single treatment session may be more than 5 differing locations, more than 25 differing locations, more than 50 differing locations, more than 100 differing locations, or more than 250 differing locations.

According to disclosed embodiments, control system 122 may use at least one actuator to selectively direct energy from the energy source (e.g., a laser such as electromagnetic radiation source 102) to different locations on target 110. In a first example, particle therapy system 100 may use least one actuator 800 to move target 110 in interaction chamber 108, and control system 122 may selectively direct energy from the energy source to differing target locations when target 110 moves (e.g., using actuator 800A). In a second example, particle therapy system 100 may use at least one actuator 800 to rotate target 110 in interaction chamber 108, and control system 122 may selectively direct energy from the energy source to differing target locations when target 110 rotates (e.g., using actuator 800A). Thus, at least some of the differing target locations may be radially spaced from each other. In a third example, particle therapy system 100 may use least one actuator 800 to change the path of electromagnetic radiation beam 104 in interaction chamber 108, and control system 122 may selectively direct energy from the energy source to differing target locations when the path of electromagnetic radiation beam 104 changes (e.g., using actuator 800B).At least one actuator 800 may cause the relative movement between target 110 and a pulsed energy beam radiating from the energy source at a rate equal to or exceeding a repetition rate of the pulsed energy beam.

Consistent with the present disclosure, target 110 may emit charged particles in response to energy application. For example, a cloud of charged particles 1006 may be emitted from target 110 in response to first pulse 1000A hitting target 110 and in response to second pulse 1000B hitting target 110. The cloud of charged particles 1006 may be captured by a solenoid (e.g., solenoid 404) that may be part of beamline 114. Beamline 114 may be used to selectively control a relative movement between particle beam 112 and patient 118 to strike a tumor 1008 with charged particles 1006 at differing tumor locations. Consistent with the present location, the term “tumor location” refers to a portion of the treatment volume (i.e., a group of cells or an area of tumor) limited in size. For example, the tumor location may be less than 5 cm³, less than 1 cm³, less than 2500 mm³, less than 50 mm³, less than 25 mm³, or less than 5 mm³. Different tumor locations may be associated with different cartesian or non-cartesian coordinates.

In some embodiments, a first bunch of charged particles 1010A may irradiate tumor 1008 at a first tumor location 1012A at time t1+ε, and a second bunch of charged particles 1010B may irradiate tumor 1008 at a second tumor location 1011B at a time t2+ε. With reference to the depicted reference system, first tumor location 1012A is located at (X=8, Y=9, Z=1), and second tumor location 1012B is located at (X=6, Y=6, Z=1). Scanning path 1014 illustrates the relative movement between particle beam 112 and patient 118 during the treatment session. In some embodiments, particle therapy system 100 may include a movable gantry for steering the beamline relative to patient 118 and for directing charged particles 1006 to differing tumor locations 1012. In addition, particle therapy system 100 may include a movable platform (e.g., support platform 116) for supporting patient 118 and a motor for moving the platform to thereby direct charged particles 1006 at differing tumor locations 1012.

The selective control of relative movement between beam of charged particles 1006 and patient 118 may be carried out in accordance with a treatment plan. The treatment plan may specify the location and size of tumor 1008 and/or the size and type of target 110. Control system 122 may coordinate the operation of the energy source and the beamline of the particle therapy system to comply with the treatment plan. Consistent with the present disclosure, in some cases there may be more possible locations of interaction on target 110 than tumor locations at tumor 1008. In a first embodiment, control system 122 may selectively direct energy from the energy source to a plurality of differing locations of interaction on target 110 to generate charged particles to treat a single tumor location. The plurality of differing locations of interaction on target 110 may be greater than three, greater than five, greater than ten, or greater than fifty. The single tumor location may be within an area of less than 5 cm², where the size of the area may be defined by a Gaussian fit of the particle distribution. Control system 122 may determine, based on the treatment plan, how to deliver charged particles from the plurality of locations of interaction to the single tumor location (e.g., scanning the tumor locations once or a couple of times). In a second embodiment, control system 122 may selectively direct energy from the energy source to a plurality of differing locations of interaction on target 110 to generate charged particles to treat multiple tumor locations. In some cases, at least one of the multiple tumor locations may receive only charged particles generated from a single location of interaction on target 110. Consistent with the present disclosure, the term “generating charged particles” means causing a plurality of charged particles to emanate from the target.

FIG. 10 further illustrates a first bunch of charged particles 1010A and a second bunch of charged particles 1010B each interacting with tumor 1008 at different depths along the Z axis. In some embodiments, the energy source may cause concurrent emission of a plurality of charged particles at multiple energy levels each time a location on the target is irradiated. Thereafter, the plurality of charged particles associated with a single particle pulse may strike the tumor at differing tumor locations. Because charged particles penetrate to different depths of a tumor depending on their energies, a single bunch of poly-energetic charged particles may penetrate the tumor such that multiple tumor locations may be concurrently treated with charged particles generated from the irradiation of a single location on the target. In the depicted example, the first bunch of charged particles 1010A may strike seven tumor locations of tumor 1008 (X=8, Z=1 to 7), and second bunch of charged particles 1010B may strike twelve tumor locations of tumor 1008 (X=6, Z=1 to 12). Control system 122 can determine the treatment depth or depths (i.e., in the Z direction) for each bunch of charged particles based on the dimensions of tumor 1008 represented in the treatment plan.

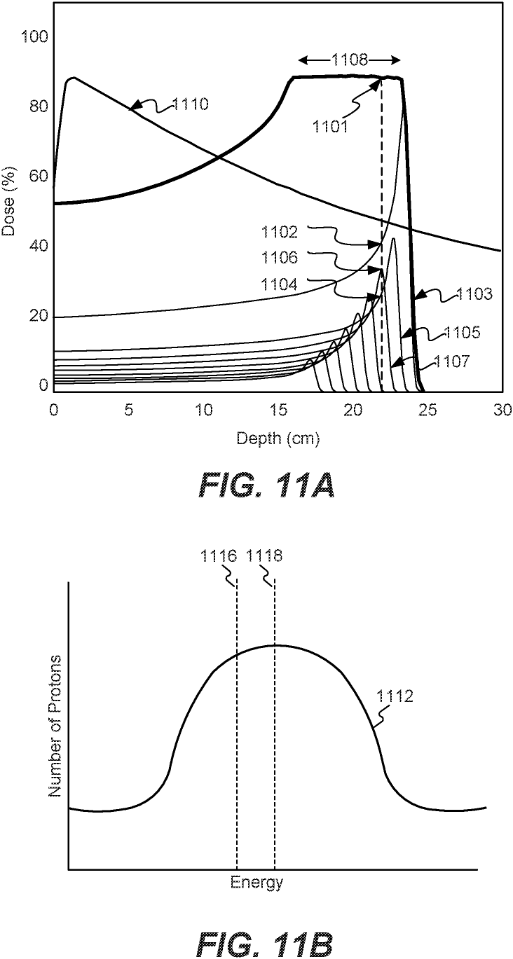

FIG. 11A shows an example plurality of pristine Bragg peaks representing energy dose profiles (e.g., Bragg curve 1103, Bragg curve 1105, and Bragg curve 1107) as a function of depth energetic particles travel in a given material (e.g., treatment volume 120). For comparison, FIG. 11 also shows a dose profile 1110 of electromagnetic radiation (e.g., X-ray or gamma ray), which has a relatively sharp rise to a maximum, followed by a gradual decrease as a function of depth. Because of this, photon-based radiation does not provide a similar range of control as charged particles like protons and carbon ions. The plurality of Bragg peaks can be combined to yield a spread-out Bragg peak (SOBP) 1108 in a cumulative dose profile. For example, referring to FIG. 11A, the dosage at point 1101 on curve 1103 (i.e., at about 22 cm depth) may be the sum of the dosages at that same depth represented by Bragg curves 1102, 1104, 1105, 1106, and 1107.

A plurality of pristine Bragg peaks can be achieved by subjecting a treatment volume to at least one bunch of charged particles having different energies. For example, as discussed above, the energy source may cause concurrent emission of a plurality of charged particles at multiple energy levels each time a single location on the target is irradiated. Control system 122 may enable the plurality of charged particles to treat the tumor with a dose characterized by a spread-out Bragg peak, the dose being delivered using only particles generated concurrently in response to an irradiation of the target. The plurality of charged particles associated with a particle bunch may irradiate the tumor at different depths. The resulting spread-out Bragg peak 1108 can be selected to overlap with the depth boundaries of treatment volume 120. For example, when the beam energies are properly selected, the spread-out Bragg peak can fall off sharply beyond the distal boundary of target volume 120.

Properly matching the depth boundaries of a target region with a spread-out Bragg peak is a consideration in particle therapy. If the distal portion of the spread-out Bragg peak is too deep, unnecessary and harmful irradiation may be provided to a region beyond the distal boundary of target volume 120 (e.g., to healthy tissue behind a tumor). If the proximal portion of the spread-out Bragg peak is too shallow, unnecessary extra radiation dose may be provided to a region in front of the proximal boundary of the target volume 120 (e.g., to healthy tissue in front of a tumor). Similarly, a proximal portion of the spread-out Bragg peak that is too deep and/or a distal portion of the spread-out Bragg peak that is too shallow may result in certain portions of target volume 120 not being irradiated properly (e.g., less than a desired amount). In some embodiments, control system 122 may determine a desired spread-out Bragg peak for each bunch of charged particles based on the treatment plan for treatment volume 120. For example, the desired spread-out Bragg peak for at least one bunch of charged particles directed to a point (X,Y) may be determined in view of the range of Z values associated with treatment volume 120 at that location.

Consistent with the present disclosure, system 100 may deliver charged particles to multiple differing locations within a treatment volume when a single location on a target is irradiated by an electromagnetic radiation beam. The electromagnetic radiation beam causes concurrent emission of a plurality of charged particles having different energies each time a location on the target is irradiated. FIG. 11B illustrates an exemplary energy profile of a particle bunch 1112 generated from a single location of interaction on target 110. To obtain the desired spread-out Bragg peak for treating a tumor located at a particular depth within a patient, a particular energy band may be needed. System 100 may filter particle beam 112 to deliver particles having the desired energy spread to the patient and eliminate particles having other energies from the particle beam. For example, to deliver particles having energies between energy 1116 and energy 1118, system 100 may filter the particle bunch by removing any charged particles having energies less than energy 1116 and greater than energy 1118. In disclosed embodiments, the multiple energy levels of a single particle bunch generated when a single location on target 110 is irradiated may span more than 5 MeV, more than 10 MeV, more than 15 MeV, more than 20 MeV, or more than 50 MeV.

Consistent with the present disclosure, control system 122 may enable selection of a subset of the multiple energy levels that conform with a treatment plan and associated with the desired spread-out Bragg peak. For example, selection of a subset of the multiple energy levels may include applying a band-pass filter to the plurality of charged particles. Such selection may be achieved by combining certain particle beam adjustment components of beamline 114. For example, beamline 114 may manipulate particle beam 112 such that charged particles having certain energies may be diverted along a different trajectory than particles having other energies. This may be achieved in a number of ways. For example, beamline 114 may include a band-pass filter to isolate charged particles having energies between energy 1116 and energy 1118. In another embodiment, beamline 114 may include a high pass filter to isolate particles having energies greater than an energy cut-off, such as energy 1116 or 1118. In another embodiment, beamline 114 may include as a low pass filter to isolate particles having energies less than an energy cut-off, such as energy 1116 or 1118. Additional details for this embodiment are provided below.

The above embodiments may be combined, and more than one filter may be used. A low pass filter and a high pass filter may be combined in series, for example, to create a band-pass filter. In such an embodiment, the low pass filter may be configured to isolate particles having energies less than energy 1118, and the high pass filter may be configured to isolate particles having energies greater than energy 1116. This may be particularly advantageous for selecting particles within a narrow energy band, especially an energy band narrower than a stand-alone band-pass filter can accommodate.

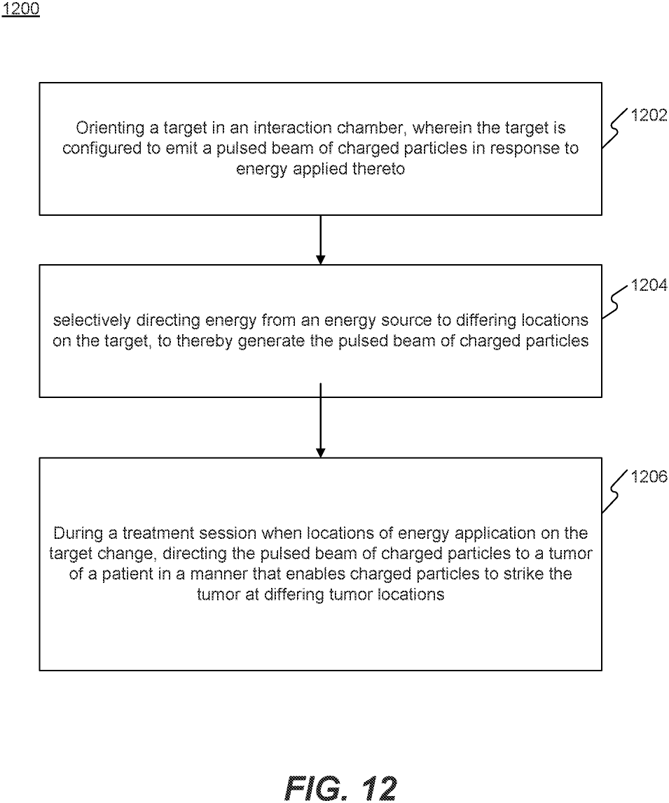

FIG. 12 is a flowchart of an example process 1200 for charged particle treatment executed by a processing device of control system 122 according to embodiments of the present disclosure. For purposes of illustration, in the following description, reference is made to certain components of particle therapy system 100. It will be appreciated, however, that other implementations are possible and that any combination of components or devices may be utilized to implement the exemplary method. It will also be readily appreciated that the illustrated method can be altered to modify the order of steps, delete steps, or further include additional steps, such as steps directed to optional embodiments.

Disclosed embodiments may include “orienting a target in an interaction chamber, wherein the target is configured to emit a pulsed beam of charged particles in response to energy applied thereto.” As discussed earlier, the pulsed beam of charged particles may include at least about 3×10⁶ positively charged particles per second (e.g., protons or ions). Alternatively, the pulsed beam of charged particles may include at least about 3×10⁶ negatively charged particles per second (e.g., electrons). By way of example only, according to step 1202 in FIG. 12 the processing device may orient target 110 in interaction chamber 108, wherein target 110 may be configured to emit a pulsed beam of charged particles 112 in response to energy applied thereto. In a specific embodiment, target 110 may contain a plurality of microstructured elements thereon, and applying the energy on each location on target 110 may destroy one or more microstructured elements at the location of interaction. This embodiment is discussed in greater detail with reference to FIG. 13 below.

Disclosed embodiments may further include “selectively directing energy from an energy source to differing locations on the target, to thereby generate the pulsed beam of charged particles.” As discussed earlier, the energy source may include electromagnetic radiation source 102, which may include a laser. By way of example only, according to step 1204 in FIG. 12, the processing device may selectively direct energy from electromagnetic radiation source 102 to differing locations on target 110, to thereby generate pulsed beam of charged particles 112. The processing device may selectively direct energy from the energy source to non-overlapping locations on target 110. Additionally or alternatively, the processing device may selectively direct energy from the energy source to partially overlapping locations on target 110. The processing device may determine whether to direct energy to non-overlapping locations on target 110 or to direct partially overlapping locations on target 110 based on the treatment plan associated with treatment volume 120. As discussed above, selectively directing energy from the energy source may include causing a change in the path of electromagnetic radiation beam 104, causing translation movement and/or rotation movement of target 110, or any combination thereof. In one embodiment, the processing device may control at least one actuator for moving target 110 in interaction chamber 108. Specifically, the processing device may selectively direct energy from the energy source to differing target locations when target 110 moves. The at least one actuator may cause target 110 to rotate such that at least some of the differing target locations are radially spaced from each other. For example, spiral path 1004 illustrates possible target locations when target 110 moves during a treatment session. In some embodiments, the at least one actuator may cause the relative movement between target 110 and a pulsed energy beam radiating from the energy source at rate equal to or exceeding a repetition rate of the pulsed energy beam. For example, the repetition rate of the pulsed energy beam may be at least about 20 Hz. Additional details on this embodiment are described above with reference to FIGS. 7 through 9.

Disclosed embodiments may further include, “during a treatment session when locations of energy application on the target change, directing the pulsed beam of charged particles to a tumor of a patient in a manner that enables charged particles to strike the tumor at differing tumor locations.” As discussed above, directing the pulsed beam of charged particles may be implemented by beamline 114. Beamline 114 may include a movable gantry for steering the beamline relative to patient 118 and/or for directing the charged particles at differing tumor locations. Additionally or alternatively, system 100 may include a movable platform for supporting patient 118 and an associated motor for moving the platform to thereby direct the charged particles at differing tumor locations. By way of example only, according to step 1206 in FIG. 12, the processing device may direct pulsed beam of charged particles 112 to treatment volume 120 in a manner that enables charged particles to irradiate tumor 1008 at differing tumor locations 1012. In this embodiment, adjusting the direction of a pulsed beam of charged particles may take place during a treatment session when the locations of interaction on target 110 change. Alternatively, a processing device may selectively direct energy from the energy source (e.g., electromagnetic radiation source 102) to a plurality of differing target locations while coordinating control of the beamline so that charged particles irradiate the tumor more than once at the same location of the X-Y plane before relocating the particle beam. For example, the same location may be within an area of the X-Y plane less than about 5 cm², where the size of the area is defined by a Gaussian fit of the particle distribution. In certain embodiments, a processing device may selectively direct energy from the energy source to at least 50 different locations on the target during a treatment session. Further, the processing device may selectively direct energy from the energy source to a plurality of differing locations on the target to generate charged particles to treat a single tumor location on the X-Y plane. The plurality of differing locations on target 110 used to treat a single tumor location (e.g., in the X-Y plane) may be at least 3 locations, at least 5 locations, at least 10 locations, or at least 25 locations.

Consistent with the present disclosure, the energy source is configured to cause concurrent emission of a plurality of charged particles at multiple energy levels each time a location on the target is irradiated. The multiple energy levels may span more than 5 MeV, more than 10 MeV, more than 20 MeV, or more than 50 MeV. The graph depicted in FIG. 11B illustrates an exemplary energy profile of a bunch of charged particles generated from a single location of interaction on target 110. The energy profile of the bunch of charged particles may be based on the type of target 110, different parameters of the energy source (e.g., electromagnetic radiation source 102), such as wavelength, intensity, temporal pulse shape (e.g., pulse width), spatial size and energy distribution, polarization, or other properties of the energy source. The processing device may cause the plurality of charged particles at the multiple energy levels to irradiate tumor 1008 at multiple differing tumor locations 1012 when a single location 1002 on target 110 is irradiated. Additionally, the processing device may enable selection of a subset of the multiple energy levels that conforms with a treatment plan for tumor 1008. In some cases, the selection of a subset of multiple energy levels may include applying a band-pass filter to the plurality of charged particles. According to one embodiment, the processing device may select a first subset of the multiple energy levels for a first bunch of charged particles and select a second subset of the multiple energy levels for a second bunch of charged particles. For example, the first subset of multiple energy levels span 5 MeV, and the second subset of multiple energy levels span 7.5 MeV.

### A Particle Therapy System That Generates an Electron Cloud on a Target

A desirable particle therapy system should produce sufficient charged particles (e.g., protons and ions) for effective treatment. For instance, a particle therapy system may deliver millions of charged particles per second to finish a treatment session within a reasonable time period. One way the disclosed particle therapy system can improve the charged particle generation rate is by causing an electron cloud to form in a region near the target before the main laser-target interaction takes place. Such an electron cloud helps the electromagnetic radiation beam to deliver more energy to the target, thereby enabling more charged particles to emanate from the target.

In disclosed embodiments, a particle therapy system (e.g., system 100) may include an interaction chamber (e.g., interaction chamber 108) configured to contain a target (e.g., target 110) having a surface with a plurality of regions thereon. The particle therapy system may also include at least one energy source (e.g., a laser such as electromagnetic radiation source 102) for supplying energy to the target. The particle therapy system may additionally include at least one processor (e.g., control system 122) configured to cause at least one energy source to deliver energy to the target in a manner that causes an electron cloud with a particle density between about 10¹⁵ to 10²¹ cm⁻³ to form in the vicinity of at least one region of the target. Thereafter, the processor (or processors) may cause the energy source (or energy sources) to irradiate the target while the electron cloud remains in the vicinity of the target, to thereby cause charged particles to emanate from the target. The particle therapy system may further include a beamline (e.g., beamline 114) configured to deliver the plurality of charged particles to a patient (e.g., patient 118).

FIG. 13 illustrates an exemplary process for causing a plurality of charged particles to emanate from target 110. In disclosed embodiments, the particle therapy system may include at least one energy source configured to deliver energy to target 110. The disclosed particle therapy system may include at least one processor configured to regulate at least one energy source to deliver a first dose of energy that causes an electron cloud to form near the target and to deliver a second dose of energy that causes a plurality of charged particles to emanate from the target. In a first example, the at least one energy source may include a single source configured for generating the first and second doses of energy (e.g., electromagnetic radiation source 102). In a second example, the at least one energy source may include a first source for generating the first dose of energy and a second source for generating the second dose of energy. The second source may be different from the first source (e.g., electromagnetic radiation sources 102A and 102B, as shown in FIG. 16). For example, the second source may be located separately from the first source, may deliver differing type of energy, or may deliver the energy through different means. For example, the second source may be any device configured to generate a pulsed particle beam. In some embodiments, the particles generated by the second source (e.g., electrons, protons, negative-H, or even neutrals) may be charged or uncharged and may have energy between several eV to keV.

Diagram 1300 depicts target 110 before any energy is delivered to it. In the illustrated example, the at least one energy source is electromagnetic radiation source 102, and the energy is delivered by electromagnetic radiation beam 104. The energy may be delivered to target 110 that includes a plurality of microstructured elements 1302. Each of the microstructured elements 1302 extends away from a surface of target 110 and is separated from other the microstructured elements 1302. As shown, electromagnetic radiation beam 104 includes a first dose of energy 1304 (e.g., a preliminary pulse) and a second dose of energy 1306 (e.g., a main pulse). Both of the pulses are directed to substantially the same location of interaction 1308 on target 110. Second dose of energy 1306 may be delivered to target 110 between about 1 to 26 ns after the first dose of energy 1304 is delivered to target 110.

Diagram 1310 depicts target 110 after a first dose of energy 1304 is delivered to target 110 and before a second dose of energy 1306 is delivered. In disclosed embodiments, the delivery of first dose of energy 1304 causes an electron cloud 1312 to form with a particle density of between about 10¹⁵ to 10²¹ cm⁻³ in a vicinity of at least one region 1314 of target 110. Region 1314 of target 110 may include one or more microstructured elements 1302. For example, the first dose of energy 1304 may have an energy flux on target 110 that is less than about 10 J/cm² and may cause an electron cloud to form with a particle density between about 10¹⁷ to 10¹⁹ cm⁻³ within about 1 µm from the surface of target 110. In some cases, electron cloud 1312 may be formed such that it surrounds at least a portion of one microstructured element 1302 of region 1314. The portion of the microstructured element surrounded by the electron cloud may be, for example, more than 10% of the microstructured element, more than 30% of the microstructured element, more than 50% of the microstructured element, or more than 75% of the microstructured element. Consistent with the present disclosure, a first dose of energy may cause ablation of at least one microstructured element 1302. The term “ablation” as used in this disclosure refers any process comparable the one illustrated in diagram 1310. For example, it may refer to the volatilization and/or ionization of a small amount of material in a region 1314, resulting in gaseous and/or particulate matter (e.g., an electron cloud or plasma) within the vicinity of an irradiated region of target 110.

Diagram 1320 depicts target 110 as a second dose of energy 1306 is delivered and electron cloud 1312 remains in the vicinity of region 1314, thereby creating energetic release of particles 1322. Consistent with the present disclosure, second dose of energy 1306 may have an intensity of about 10¹⁸ W/cm² or more, about 10¹⁹ W/cm² or more, about 10²⁰ W/cm² or more, or about 10²¹ W/cm² or more. In some cases, second dose of energy 1306 may be at least 500 times more energetic than first dose of energy 1304, at least 1000 times more energetic than first dose of energy 1304, at least 1500 times more energetic than first dose of energy 1304, at least 2000 times more energetic than first dose of energy 1304, or at least 2500 times more energetic than first dose of energy 1304. As mentioned above, delivering second dose of energy 1306 may destroy certain microstructured elements 1302 in region 1314.

The term “destroy” as used in this disclosure refers any process comparable the one illustrated in diagram 1330. For example, it may refer to the total eradication of at least part of the structure (or structures) in a region 1314, resulting in charged particles suitable for forming an ion beam emanating from within the vicinity of an irradiated region of target 110. Diagram 1330 depicts target 110 after second dose of energy 1306 is delivered to target 110. In disclosed embodiments, delivering second dose of energy 1306 while electron cloud 1312 remains in the vicinity of region 1314 causes a plurality of charged particles 1332 to emanate from target 110. Consistent with the present disclosure, delivering second dose of energy 1306 may cause at least 3×10⁶ charged particles to emanate from target 110 per delivery or at least 3×10⁸ charged particles to emanate from target 110 per second. The disclosed particle therapy system may further include a beamline (e.g., beamline 114) configured to deliver the plurality of charged particles 1332 to patient 118. In some embodiments, the plurality of charged particles 1332 may exhibit a large divergence. As an example, the beam size of a plurality of charged particles 1332 emitted from target 110 may expand by a factor of about 100 over a short distance, such as 1 cm. Accordingly, beamline 114 may include a solenoid for reducing the divergence of the particle beam.

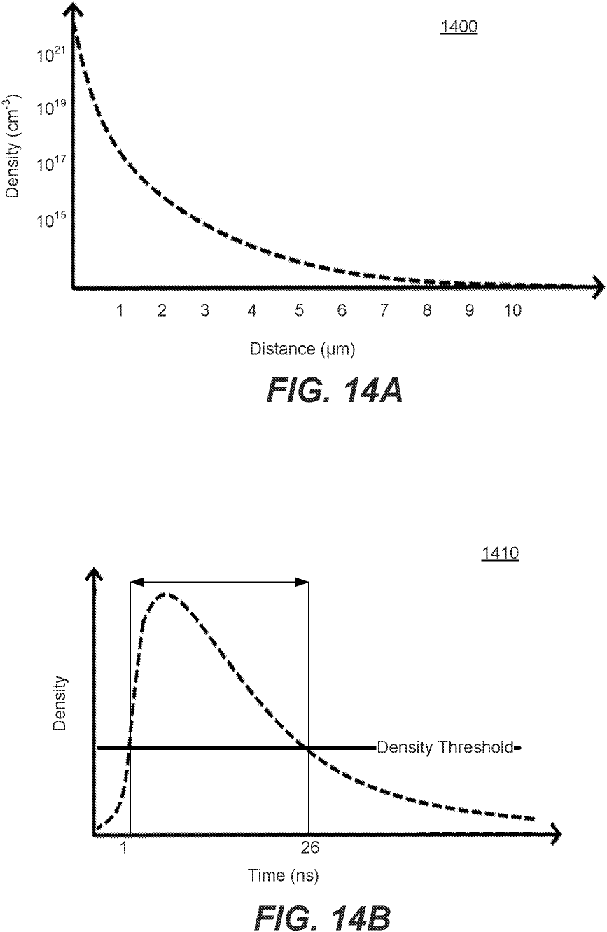

FIG. 14A illustrates an example scenario of the density of electron cloud 1312 versus distance from a microstructured element 1302 a short time after delivering first dose of energy 1304 but before delivering second dose of energy 1306. A person skilled in the art would recognize that the numbers in graph 1400 are just exemplary and may vary significantly based on the type of target and the energy flux of first dose of energy 1304. According to disclosed embodiments, electron cloud 1312 may have a particle density of between about 10¹⁵ and 10²¹ cm⁻³ in a vicinity of region 1314 of target 110. In the illustrated example, the vicinity of region 1314 of target 110 may be less than about 3 µm, and the particle density of electron cloud 1322 may be about 10¹⁷ cm⁻³ at a distance of about 1 µm. In other cases, the vicinity of region 1314 of target 110 may be less than 10 µm, less than 7.5 µm, or less than 5 µm. In addition, the particle density vs. distance relationship may differ based on the target’s composition and shape and/or the first dose of energy 1304.

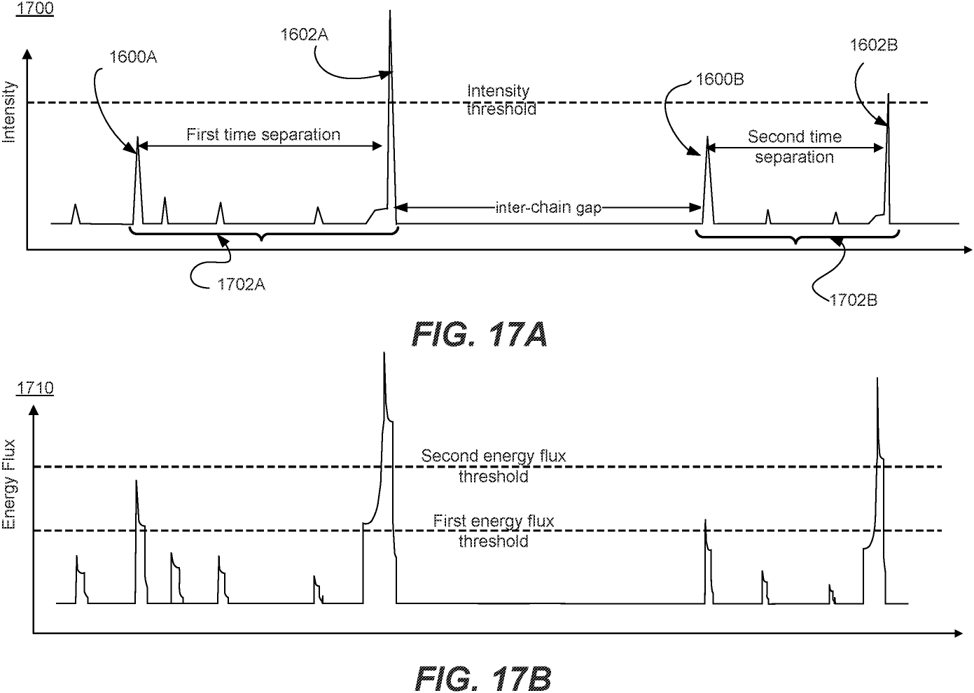

FIG. 14B illustrates the variation of particle density at a fixed location in electron cloud 1312 over time. The graph starts when first dose of energy 1304 is delivered to target 110. Consistent with the present disclosure, second dose of energy 1306 may be delivered to target 110 between about 1 to 26 ns after first dose of energy 1304 is delivered to target 110. For example, second dose of energy 1306 may be delivered to target 110 between about 1 to 20 ns, between about 2 to 18 ns, or between about 5 to 25 ns after first dose of energy 1304 is delivered to target 110. In some embodiments, second dose of energy 1306 may be delivered to target 110 when the particle density of electron cloud 1312 in a vicinity of region 1314 is greater than a threshold value. The threshold value may depend on various parameters, including the composition, shape, and orientation of target 110, the intensity of second dose of energy 1306, the desired energy levels of charged particles 1332, or other relevant parameters. In some examples, the particle density threshold may be about 10¹⁵ cm⁻³, and a time separation between first dose 1304 and second dose 1306 may be based on an estimated particle density of electron cloud 1322. In some embodiments, particle therapy system 100 may include a filter configured to prevent, during a time separation between the first dose and the second dose, delivering any dose of energy with an energy flux greater than about 0.1 J/cm². Such embodiments are discussed below with reference to FIGS. 17A and 17B.

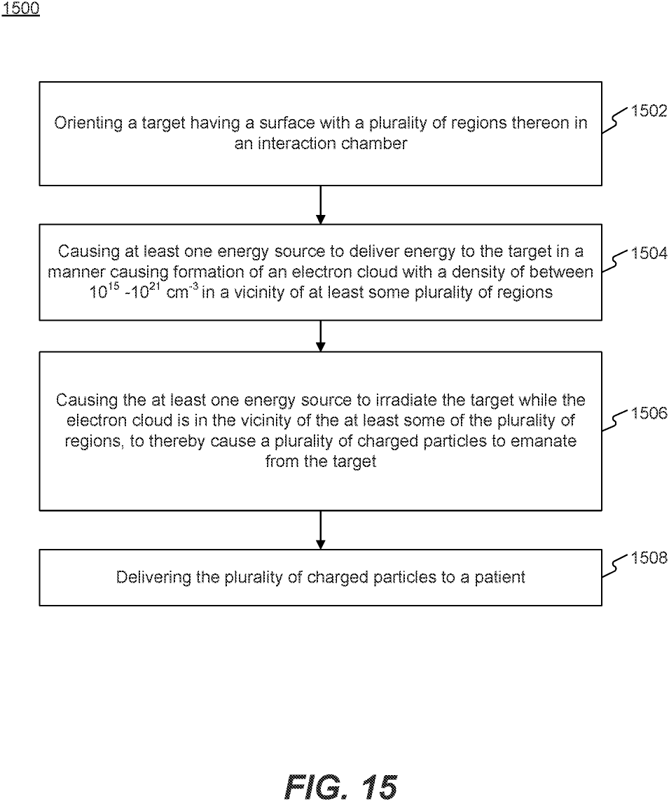

FIG. 15 is a flowchart of an example process 1500 for particle therapy executed by a processing device of control system 122 according to embodiments of the present disclosure. For purposes of illustration in the following description, reference is made to certain components of particle therapy system 100. It will be appreciated, however, that other implementations are possible and that any combination of components or devices may be utilized to implement the exemplary method. It will also be readily appreciated that the illustrated method can be altered to modify the order of steps, delete steps, or further include additional steps, such as steps directed to optional embodiments.

Disclosed embodiments may include “orienting a target having a surface with a plurality of regions thereon in an interaction chamber.” As discussed earlier, a target may include a plurality of microstructured elements, and each region of the target may include at least one microstructured element. By way of example only, according to step 1502 in FIG. 15, the processing device may orient target 110 having a surface with a plurality of regions 1314 thereon in interaction chamber 108. In some embodiment, orienting target 110 may include positioning target 110 along the path of electromagnetic radiation beam 104. Orienting target 110 may include causing a relative movement between target 110 and electromagnetic radiation beam 104 at a speed associated with a repetition rate of electromagnetic radiation source 102. In other cases, particle therapy system 100 may include securing target 110 at a fixed position pointing to the energy source.

Disclosed embodiments may include “causing at least one energy source to deliver energy to the target in a manner causing formation of an electron cloud with a density of between 10¹⁵ -10²¹ cm⁻³ in a vicinity of at least some plurality of regions.” As discussed above, an electron cloud may surround at least a portion of one or more microstructured elements of target 110. By way of example only, according to step 1504 in FIG. 15, the processing device may cause at least one energy source to deliver energy to target 110 in a manner that causes electron cloud 1312 to form with a particle density between about 10¹⁵ and 10²¹ cm⁻³ in a vicinity of at least one region 1314. For example, a processing device may cause an electron cloud 1312 to form with a particle density between about 10¹⁷ and 10¹⁹ cm⁻³ within about 1 µm of target 110 (e.g., within about 1 µm of at least one microstructured element of target 110).

A person skilled in the art would recognize that the particle density of electron cloud 1312 may depend on the relativistic critical density (n_{crel}) and the wavelength (λ) associated with first dose of energy. For example, the electron-cloud density (nₑ) of electron cloud 1312 may be in the range: n_{crit}/10⁶ < nₑ ≤ n_{crit}, where n_{crit} is the critical plasma density related to the wavelength (λ) of the energy source that provided the first dose of energy (e.g., electromagnetic radiation source 102). The critical plasma density may be defined by the following equation:

$${n}_{c r i t} = {( 2 π c / e )}^{2} ( {ε}_{o} {m}_{e}^{*} / {λ}^{2} )$$

where c is the speed of light in vacuum, e is the electronic charge, εₒ is the free-space permittivity, and

$${m}_{e}^{*}$$

is the relativistic mass of the electron. The relativistic electron mass is related to the rest mass (mₑ) of the electron via

$${m}_{e}^{*} = {m}_{e} / \sqrt{1 - {\left(v / c\right)}^{2}} ,$$

where v is the electron velocity. The relativistic electron mass becomes larger than electron rest mass at high laser intensities (>10²⁰ W/cm²), leading to relativistic transparency, which may play a role in enhancing the particles energies. A typical scale length (lₛ) of electron cloud 1312 may be linked to the wavelength (λ) of the energy source that provided the first dose of energy and may be in the range of: λ/2π ≤ lₛ < 10λ, where λ/2π is the plasma skin depth at n_{crit} up to which an electromagnetic radiation can penetrate. In a first example, electromagnetic radiation source 102 may be associated with λ= 800 nm, such that the electron-cloud density (nₑ) of electron cloud 1312 may be about 10¹⁵ < nₑ ≤ 10²¹ and a typical scale length of electron cloud 1312 may be about 0.1 µm ≤ lₛ < 10 µm. In a second example, the energy source may be associated with λ= 400 nm, such that the electron cloud density (nₑ) of electron cloud 1312 may be about 10¹⁶ < nₑ ≤ 10²², and the typical scale length of electron cloud 1312 may be about 0.05 µm ≤ lₛ < 5 µm. In a third example, the energy source may be associated with λ= 1600 nm, such that the electron cloud density (nₑ) of electron cloud 1312 may be about 10¹⁴ < nₑ ≤ 10²⁰, and a typical scale length of electron cloud 1312 may be about 0.2 µm ≤ lₛ < 20 µm.

Consistent with the present disclosure, the at least one energy source may include a source of electromagnetic radiation (e.g., electromagnetic radiation source 102), and the processing device may regulate the at least one energy source to deliver a first dose with an energy flux on the target less than about 10 J/cm². In some embodiments, target 110 may have a surface on which microstructured elements 1302 are disposed, and first dose of energy 1304 may be configured to cause ablation of one or more microstructured element. In contrast, second dose of energy 1306 may be configured to destroy at least part of the one or more microstructured elements.

Disclosed embodiments may include “causing the at least one energy source to irradiate the target while the electron cloud is in the vicinity of the at least some of the plurality of regions, to thereby cause a plurality of charged particles to emanate from the target.” A single power source may cause an electron cloud to form in a vicinity of the target and cause a plurality of charged particles to emanate from the target. Alternatively, one power source may cause an electron cloud to form in a vicinity of the target, whereas a different power source causes a plurality of charged particles to emanate from the target. By way of example only, according to step 1506 in FIG. 15, a processing device may cause the at least one energy source to irradiate target 110 while electron cloud 1312 is in a vicinity of one or more regions 1314, to thereby cause a plurality of charged particles 1332 to emanate from target 110. Consistent with the present disclosure, the processing device may regulate the at least one energy source to deliver first dose of energy 1304 and cause electron cloud 1312 to form and to deliver second dose of energy 1306 and cause a plurality of charged particles 1332 to emanate from target 110. In some embodiments, the processing device may regulate the at least one energy source to deliver second dose of energy 1306 with an intensity on target of at least about 10¹⁸ W/cm². For example, the processing device may regulate the at least one energy source to deliver second dose of energy 1306 in an amount that is about 1000 times or more energetic than first dose of energy 1304. In addition, the processing device may cause second dose of energy 1306 to be delivered to target 110 between about 1 to 26 ns after first dose of energy 1304 is delivered to target 110. In some embodiments, particle therapy system 100 may include a filter configured to prevent, during a time separation between first dose 1304 and second dose 1306, delivering any dose of energy with an energy flux greater than about 0.1 J/cm^{2.}

Disclosed embodiments may further include “delivering the plurality of charged particles to a patient.” The plurality of charged particles emanating from target 110 in response to delivering a second dose of energy 1306 may have different energy levels. The various energy levels of the charged particles emanating from target 110 in response to second dose of energy 1306 may span for more than about 5 MeV. By way of example only, according to step 1508 in FIG. 15, a processing device may cause delivery of charged particles 1332 to patient 118 using beamline 114. Consistent with the present disclosure, the processing device may regulate the delivery of energy to target 110 in a manner that causes at least 3×10⁶ charged particles per delivery. In some embodiments, regulating the delivery of energy to target 110 may include causing a relative movement between target 110 and electromagnetic radiation beam 104 at a speed associated with a repetition rate of electromagnetic radiation source 102, to thereby vary a location of interaction of pulsed electromagnetic radiation beam 104 on the surface of target 110 and thereby cause a resultant emission from target 110 of at least 3×10⁶ charged particles per second dose of energy or at least 3×10⁸ charged particles per second.

In certain embodiments, discussed below with reference to FIGS. 16 through 18, forming electron cloud 1312 with a particle density of between 10¹⁵ and 10²¹ cm⁻³ may be caused by directing a preliminary pulse of electromagnetic radiation beam 104 towards target 110, and the emanation of charged particles 1332 may be caused by directing a main pulse of electromagnetic radiation beam 104 towards target 110. In one example, the preliminary pulse may exceed an energy flux threshold and have an energy flux of between about 0.1 and 10 J/cm², and the main pulse may have an intensity of at least about 10¹⁸ W/cm².

### A Particle Therapy System That Uses Preliminary Pulse Prior to Main Pulse

The following disclosure describes a specific example of a particle therapy system that generates an electron cloud prior to a laser-target interaction. In this example, at least one electromagnetic radiation source generates an electron cloud and causes a plurality of charged particles to emanate from the target. The at least one electromagnetic radiation source may produce a preliminary pulse for generating the electron cloud and thereafter produce a main pulse for causing the plurality of charged particles to emanate from the target. To maximize the number of charged particles (e.g., protons or ions) that emanate from the target, the particle therapy system controls both the preliminary pulse and the main pulse according to certain requirements.

In disclosed embodiments, a particle therapy system (e.g., system 100) may include an interaction chamber (e.g., interaction chamber 108) configured to contain a target (e.g., target 110). The particle therapy system may also include at least one electromagnetic radiation source (e.g., electromagnetic radiation source 102). The particle therapy system may additionally include at least one processor (e.g., control system 122) configured to cause a pulsed beam of charged particles to be emitted from the target by regulating the at least one electromagnetic radiation source to irradiate the target with a plurality of pulse chains, each pulse chain including a preliminary pulse and a main pulse. The plurality of pulse chains may comply with the following conditions:
The preliminary pulse exceeds an energy flux threshold and has an energy flux on the target of between about 0.1 and 15 J/cm².
The main pulse has an intensity on the target of at least about 10¹⁸ W/cm².
A time separation between the preliminary pulse and the main pulse is between about 0.5 ns and 50 ns.
During the time separation between the preliminary pulse and the main pulse, the target is free from irradiation exceeding an energy flux threshold.
The particle therapy system may also include a magnetic beamline (e.g., beamline 114) for delivering the pulsed beam of charged particles (e.g., particle beam 112) to a patient (e.g., patient 118).

Consistent with the present disclosure, particle therapy system 100 may generate a preliminary pulse to ablate at least a portion of the target so that a subsequent main pulse may interact with an ablated location of interaction at target 110. Doing so enhances the acceleration of protons and other charged particles in the vicinity of that location. After the preliminary pulse, the ablated material (e.g., electron cloud 1312) and associated plasma (e.g., plasma generated in response to preliminary pulse) expand. As the ablated material expands, the main pulse arrives at a time that enables it to further ionize the target material and interact with the pre-ionized plasma to produce energetic particles. The electromagnetic radiation source (or sources) generating the preliminary and the main pulses may have fixed-pulse configurations or may vary parameters, such as energy flux, temporal duration, wavelength, timing, or others. In some embodiments, a single electromagnetic radiation source 102 may generate the preliminary and the main pulses. For example, optics 106 may include one or more components for extracting the preliminary pulse from the main pulse and for generating the required time separation.

In alternative embodiments, two different electromagnetic radiation sources may generate the preliminary and the main pulses. Reference is now made to FIG. 16, which illustrates such an embodiment. First electromagnetic radiation source 102A and second electromagnetic radiation source 102B generate the preliminary and the main pulses, respectively. First electromagnetic radiation source 102A generates preliminary pulses 1600 (e.g., preliminary pulse 1600A and preliminary pulse 1600B), and second electromagnetic radiation source 102A generates main pulses 1602 (e.g., main pulse 1602A and main pulse 1602B). Control system 122 may coordinate the operation of first electromagnetic radiation source 102A and second electromagnetic radiation source 102B with the movement of target 110 such that pairs of preliminary pulse 1600 and main pulse 1602 interact with substantially the same location at target 110. Consistent with the present disclosure, the location of interaction of preliminary pulse 1600 and the location of interaction of main pulse 1602 may have more than 50% overlap, for example, more than 75% overlap, more than 90% overlap, or more than 95% overlap. In one embodiment, preliminary pulse 1600A and main pulse 1602A may interact with a first location of interaction, and preliminary pulse 1600B and main pulse 1602B may interact with a second location of interaction that differs from and does not overlap with the first location of interaction.

As discussed above with reference to FIG. 13, target 110 may have a surface on which microstructured elements are disposed (e.g., microstructured elements 1302). In some embodiments, upon interaction with target 110, preliminary pulse 1600 (in the discussion above referred as a “first dose of energy”) may ablate at least some microstructured elements 1302, and upon interacting with target 110, main pulse 1602 (in the discussion above referred as a “second dose of energy”) may destroy at least part of the one or more microstructured elements 1302. Further, at least one electromagnetic radiation source 102 (e.g., electromagnetic radiation source 102A and second electromagnetic radiation source 102B) may irradiate target 110 with preliminary pulse 1600 to generate an electron cloud with a particle density of between about 10¹⁷ and 10¹⁹ cm⁻³ in the vicinity of at least part of target 110. For example, at least one electromagnetic radiation source 102 may generate an electron cloud within about 1 µm of target 110. At least one electromagnetic radiation source 102 is further configured to irradiate target 110 with main pulse 1602 while the electron cloud remains in the vicinity of target 110, to cause a plurality of charged particles (e.g., particles 1332) to emanate from target 110.

FIG. 17A illustrates the energy of pulses generated by at least one electromagnetic radiation source 102 over time. In disclosed embodiments, control system 122 may cause a pulsed beam of charged particles (e.g., particle beam 112) to be emitted from target 110 by regulating at least one electromagnetic radiation source 102 to irradiate target 110 with a plurality of pulse chains 1702. Each pulse chain may include a preliminary pulse 1600 and a main pulse 1602. The width (e.g., the full width at half maximum) of a pulse generated by at least one electromagnetic radiation source 102 is not limited. In some cases, the pulse width may be in the nanoseconds range. For example, at least one electromagnetic radiation source 102 may generate pulses with a width of less than one nanosecond, such as about a tenth of a nanosecond, about ten picoseconds, or less. At least one electromagnetic radiation source 102 may be adapted to continuously generate pulses with fixed gaps between them or with variable gaps between them. In addition, at least one electromagnetic radiation source 102 may group a number of pulses to form a pulse chain, meaning a series of successive pulses. A pulse chain generated in accordance with the present disclosure may include a preliminary pulse, a main pulse, and additional pulses. For example, graph 1700 depicts a first pulse chain 1702A with preliminary pulse 1600A, main pulse 1602A, and three additional small pulses between them. Graph 1700 also depicts a second pulse chain 1702B with preliminary pulse 1600B, main pulse 1602B, and two additional small pulses between them.

The pulse chain may be characterized in that the time duration between the preliminary pulse and main pulse, referred to herein as “time separation” or “intra-chain gap,” is different from the duration between pulse chains, referred to herein as “inter-chain gap.” Typically, the inter-chain gap may be greater than the intra-chain gap. For example, the inter-chain gap may be more than 10 times the intra-chain gap, more than 50 times the intra-chain gap, or more than 100 times the intra-chain gap. In a first example, the inter-chain gap may be between about 70 ns and about 200 ns, and the intra-chain gap may be between about 0.5 ns and about 50 ns. In a second example, the inter-chain gap may be between about 50 ns and about 250 ns, and the intra-chain gap may be between about 1 ns and about 25 ns. In a third example, the inter-chain gap may be greater than about 50 ns, and the intra-chain gap may be between about 2 ns and about 15 ns. The optimal timing of the intra-chain gap may differ from target to target (e.g., with respect to the target’s surface features, composition, and orientation); thus, a control system may set an intra-chain gap based on data indicative of a laser-target interaction. In some embodiments, particle therapy system 100 may determine the time separation (i.e., the intra-chain gap) between preliminary pulse 1600 and main pulse 1602 based on the desired properties of the pulsed beam of charged particles. For example, in some cases, the time separation between preliminary pulse 1600 and main pulse 1602 may have an effect on the energy levels of the charged particles emitted from target 110. In the illustrated example, the first time separation (i.e., between preliminary pulse 1600A and main pulse 1602A) may be greater than the second time separation (i.e., between preliminary pulse 1600B and main pulse 1602B).

Consistent with the present disclosure, during a treatment session at least one electromagnetic radiation source 102 may generate a first main pulse (e.g., main pulse 1602A) with a first intensity at the target greater than about 10¹⁸ W/cm² and a second main pulse (e.g., main pulse 1602B) with a second intensity at the target different than the first intensity. In some embodiments, as depicted in FIG. 17A, a first preliminary pulse (e.g., preliminary pulse 1600A) that precedes a first main pulse, and a second preliminary pulse (e.g., preliminary pulse 1600B) that precedes a second main pulse, may have the same intensity at the target. The intensity of main pulse 1602 may be independent of the intensity of preliminary pulse 1600. The intensities of preliminary pulses 1600 may be fixed during a treatment session, and the values of main pulses 1602 may be adjusted based on the desired properties of the resultant charged particles. Adjustments to the main pulses may be frequent (e.g., from chain pulse to chain pulse) or occur less frequently (e.g., once per treatment session). Alternatively, the intensities and/or the energy flux of the preliminary pulses and the intensities of the main pulses may remain fixed during a full treatment session.

In disclosed embodiments, main pulse 1602 may have an intensity (I) at the target greater than an intensity threshold, and preliminary pulse 1600 may have an intensity at the target less than that intensity threshold. The intensity threshold may be about 10¹⁸ W/cm², and the intensity of main pulse 1602 at the target may be between about 10¹⁸ to 10²⁰ W/cm², between about 10²⁰ to 10²² W/cm², between about 10²² to 10²⁴ W/cm², or greater than about 10²⁴ W/cm². For example, main pulse 1602 may have an intensity on target of at least about 10²⁰ W/cm². In some cases, the intensity contrast ratio of main pulse 1602 to the one or more additional pulses other than preliminary pulse 1600 should be greater than about 10⁶. For example, chain pulse 1702A includes three additional pulses, and the intensity contrast ratio of main pulse 1602A to these additional pulses may be between about 10⁶ to 10⁸ or greater than about 10⁸. In one embodiment, any such additional pulses may have energy flux below a damage threshold of target 110. If significant additional pulses (i.e., above the damage threshold for ablation) are present, the acceleration of charged particles due to the presence of electron cloud 1312, for example, may be less efficient.

FIG. 17B illustrates the energy flux of pulses generated by at least one electromagnetic radiation source 102 over time. In disclosed embodiments, control system 122 may cause electromagnetic radiation source 102 to generate a preliminary pulse 1600 that exceeds a first energy flux threshold (e.g., damage threshold) and have an energy flux at the target between about 0.01 J/cm² to 15 J/cm², such as between about 0.1 and 10 J/cm². In some cases, the energy flux of preliminary pulse 1600 that interacts with target 110 prior to main pulse 1602 may be above about 5 J/cm². Alternatively, the energy flux on target of preliminary pulse 1600 may be between about 0.001 and 15 J/cm². If preliminary pulse 1600 has an energy flux below a first energy flux threshold, it may have no effect on the acceleration of charged particles. In addition, control system 122 may cause electromagnetic radiation source 102 to generate a preliminary pulse below a second energy flux threshold. If preliminary pulse 1600 has an energy flux above the second energy flux threshold, target 110 may be damaged, and particle acceleration may not occur when main pulse 1602 arrives. A relatively narrow window of energy flux may exist for which preliminary pulse 1600 can ablate target 110 and main pulse 1602 can produce energetic charged particles (e.g., protons, ions, or electrons). In some embodiments, preliminary pulse 1600 may have an energy flux at the target between about 1.5 and 50 times a first energy flux threshold, such as between about 2 and 20 times the first energy threshold.

The exact value of the energy flux of preliminary pulse 1600 may depend on properties of target 110 (e.g., composition, physical shape, distribution of microstructured elements, surface roughness, orientation, etc.) and on the arrival time of preliminary pulse 1600 relative to main pulse 1602. Different targets and different timing may provide for different optimal energy fluxes. In some embodiments, the temporal duration of preliminary pulse 1600 may be about the same as main pulse 1602, e.g., several tens of femtoseconds, several hundreds of femtoseconds, several pico-seconds, tens of pico-seconds, hundreds of pico-seconds, or nanoseconds. In some embodiments, the wavelength of preliminary pulse 1600 may be about the same as main pulse 1602 or a different but related wavelength relative to main pulse 1602. For example, the wavelength of preliminary pulse 1600 may be a second harmonic (½ the wavelength), third harmonic (⅓ of the wavelength), or twice the wavelength of main pulse 1602.

Particle therapy system 100 may include one or more sensors configured to measure characteristics of target 110 (e.g., surface roughness) and determine a set of properties for preliminary pulse 1600 and main pulse 1602 based on the measured characteristics of target 110. Particle therapy system 100 may use the determined set of properties to generate preliminary pulse 1600 and main pulse 1602 for generating a beam of charged particles. For example, control system 122 may determine the energy flux of preliminary pulse 1600 and the timing based on the surface roughness of the target. According to additional embodiments, control system 122 may also determine a value for a first energy flux threshold and/or a second energy flux threshold based on physical characteristics of target 110 (e.g., composition, physical shape, distribution of microstructured elements, surface roughness, orientation, etc.).

In disclosed embodiments, particle therapy system 100 may determine a set of properties for preliminary pulse 1600 and use the determined set of properties to generate preliminary pulse 1600 and main pulse 1602. In one example, the set of properties may include the energy flux of preliminary pulse 1600. In some cases, not shown in the figures, preliminary pulse 1600 may comprise a group of pulses, in which case the time separation is measured from the first pulse of the group. Such a group of pulses may include a preliminary pulse 1600 that exceeds a first energy flux threshold and has an energy flux at the target of between about 0.1 and 15 J/cm². The energy flux may be controlled by changing the number of pulses, changing the energy of each pulse, changing a timing between pulses, or a combination thereof. In another example, the set of properties may include the timing of preliminary pulse 1600, i.e., how much time exists between the preliminary pulse 1600 and main pulse 1602. The set of properties may affect the ablation of target 110 and may affect the properties of the beam of charged particles generated. In one embodiment, the energy flux of preliminary pulse 1600 at the target may be determined based on the desired properties of the pulsed beam of charged particles. For example, when treating a patient or a certain location of a tumor in the X-Y plane, a specific energy band of charged particles may be desired. The system may determine a set of properties for preliminary pulse 1600 and/or main pulse 1602 based on the desired energy level(s) or desired energy band.

A relatively narrow window of energy flux may exist for preliminary pulse 1600. If preliminary pulse 1600 (referred above as a first dose of energy 1304) has an energy flux below a first energy flux threshold, it may have no effect on accelerating the charged particles. For example, it may not form electron cloud 1312 with a particle density between about 10¹⁵ and 10²¹ cm⁻³. If preliminary pulse 1600 has an energy flux above a second energy flux threshold, target 110 may be excessively damaged before main pulse 1602 arrives. For example, one or more microstructured elements 1302 may be so damaged as to inhibit acceleration of any charged particles generated. During the time separation (e.g., the intra-chain gap), particle therapy system 100 may prevent exposing target 110 to any radiation exceeding the first energy flux threshold. Similarly, particle therapy system 100 may prevent exposing target 110 to any radiation exceeding the first energy flux threshold within a half-millisecond before arrival of preliminary pulse 1600.

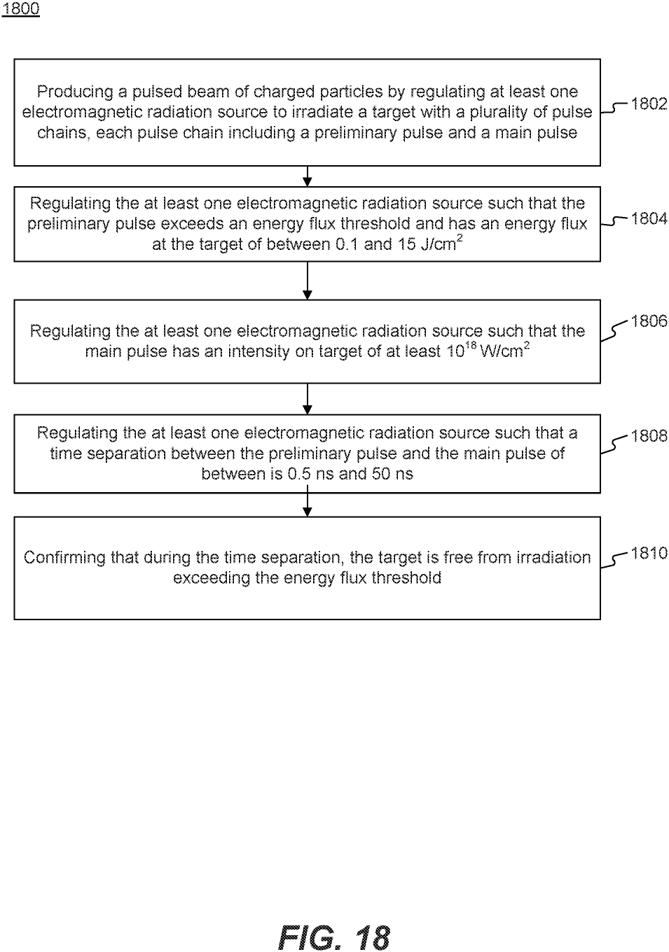

FIG. 18 is a flowchart of an example process 1800 for particle therapy executed by a processing device of control system 122 according to embodiments of the present disclosure. For purposes of illustration, in the following description, reference is made to certain components of particle therapy system 100. It will be appreciated, however, that other implementations are possible and that any combination of components or devices may be utilized to implement the exemplary method. It will also be readily appreciated that the illustrated method can be altered to modify the order of steps, delete steps, or further include additional steps, such as steps directed to optional embodiments.

Disclosed embodiments may include “producing a pulsed beam of charged particles by regulating at least one electromagnetic radiation source to irradiate a target with a plurality of pulse chains, each pulse chain including a preliminary pulse and a main pulse.” As discussed earlier, the pulsed beam of charged particles may include at least about 3×10⁶ charged particles per second, for example, at least about 3×10⁸ charged particles per second. The charged particles may be negatively charged or positively charged. By way of example only, according to step 1802 in FIG. 18, the processing device may produce a pulsed beam of charged particles (e.g., particle beam 112) by regulating at least one electromagnetic radiation source 102 to irradiate target 110 with a plurality of pulse chains 1702, each pulse chain including preliminary pulse 1600 and a main pulse 1602. Consistent with the present disclosure, the at least one electromagnetic radiation source 102 may include a first electromagnetic radiation source for generating main pulses 1602 and a second electromagnetic radiation source for generating preliminary pulses 1600. Alternatively, the at least one electromagnetic radiation source 102 may include a single electromagnetic radiation source for generating both main pulses 1602 and preliminary pulses 1600. Generating main pulses 1602 and preliminary pulses 1600 may include using dedicated optics to extract preliminary pulse 1600 from main pulse 1602.

Disclosed embodiments may include “regulating the at least one electromagnetic radiation source such that the preliminary pulse exceeds an energy flux threshold and has an energy flux on target of between 0.1 and 15 J/cm².” The electron cloud may at least partially surround a portion of one or more microstructured elements 1302 of target 110. By way of example only, according to step 1804 in FIG. 18, a processing device may regulate the at least one electromagnetic radiation source 102 such that preliminary pulse 1600 exceeds an energy flux threshold and has an energy flux at the target between about 0.1 and 15 J/cm². In some embodiments, preliminary pulse 1600 may have an energy flux at the target between about 2 and 20 times an energy flux threshold. The energy flux threshold may be between about 0.001 J/cm² to 10 J/cm². For example, preliminary pulse 1600 may have an energy flux at the target between about 0.1 and 1 J/cm². A processing device may determine the energy flux of preliminary pulse 1600 based on the desired properties of the pulsed beam of charged particles 112, such as desired energy levels of the charged particles. A processing device may also determine a value for the energy flux threshold based on physical characteristics of target 110 (e.g., composition, physical shape, distribution of microstructured elements, surface roughness, orientation, etc.)

Disclosed embodiments may include “regulating the at least one electromagnetic radiation source such that the main pulse has an intensity at the target of at least 10¹⁸ W/cm².” The main pulse may have an intensity at the target of at least about 10²⁰ W/cm². By way of example only, according to step 1806 in FIG. 18, a processing device may regulate the at least one electromagnetic radiation source such that the main pulse has an intensity at the target of at least about 10¹⁸ W/cm². In some cases, the processing device may regulate electromagnetic radiation source(s) 102 such that, during a treatment session, the at least one electromagnetic radiation source generates a first main pulse with a first intensity at the target greater than about 10¹⁸ W/cm² and a second main pulse with a second intensity at the target different than the first intensity. In these cases, a first preliminary pulse that precedes the first main pulse and a second preliminary pulse that precedes the second main pulse have the same intensity as one another at the target. In some cases, the second intensity (of the second main pulse) may be greater than the first intensity (of the first main pulse). For example, the second intensity may be at least 1.25 times greater than the first intensity. In such cases, the first preliminary pulse and the second preliminary pulse may have the same energy flux as one another.

Disclosed embodiments may further include “regulating the at least one electromagnetic radiation source such that a time separation between the preliminary pulse and the main pulse is between 0.5 ns and 50 ns.” The time separation between the preliminary pulse and the main pulse may be determined based on the desired properties of the pulsed beam of charged particles. By way of example only, according to step 1808 in FIG. 18, a processing device may regulate the at least one electromagnetic radiation source such that a time separation between a preliminary pulse and main pulse is between about 0.5 ns and 50 ns. Consistent with the present disclosure, the processing device may regulate at least one electromagnetic radiation source 102 such that the determined time separation between preliminary pulse 1600 and main pulse 1602 is between about 1 ns and 26 ns or between about 2 ns to 18 ns.

Disclosed embodiments may further include “confirming that, during the time separation, the target is free from irradiation exceeding an energy flux threshold.” By way of example only, according to step 1810 in FIG. 18, a processing device may confirm that, during the time separation between preliminary and main pulses, the target is free from irradiation exceeding an energy flux threshold. Consistent with the present disclosure, a processing device may regulate the at least one electromagnetic radiation source 102 to prevent, within about a half-millisecond before the arrival of preliminary pulse 1600, energy exceeding an energy flux threshold from irradiating target 110. System 100 also may include a filter configured to prevent exposing target 110 to any radiation exceeding an energy flux threshold between the arrival of the preliminary pulse and the arrival of the main pulse.

In embodiments like those discussed with reference to FIGS. 13 through 15, a processing device may regulate the at least one electromagnetic radiation source 102 to irradiate target 110 with preliminary pulse 1600 (e.g., first dose of energy 1304) and cause an electron cloud 1312 with a particle density between about 10¹⁵ and 10²¹ cm⁻³ to partially surround at least part of target 110. The processing device may further irradiate target 110 with main pulse 1602 (e.g., second dose of energy 1306) while electron cloud 1312 remains in the vicinity of at least part of target 110, to cause a plurality of charged particles to emanate from target 110. At least one electromagnetic radiation source 102 is configured to irradiate target 110 with preliminary pulse 1600 to form an electron cloud within about 1 µm of target 110. The plurality of charged particles emanating from target 110 in response to a delivery of main pulse 1602 may have different energies, which may span a range of more than about 5 MeV, for example.

### A Particle Therapy System That Conforms Beam Characteristics to Tumor Treatment Plan

In contrast to conventional particle therapy systems that use single-energy accelerators to create substantially monoenergetic beams, the pulsed particle beam generated from target-laser interactions is polyenergetic, with a notably larger energy spread. An exemplary particle energy profile of the particle beam is depicted in FIG. 11B. A particle therapy system as described in the present disclosure has the ability to deliver to a tumor at the same time charged particles associated with multiple energy levels. Doing so enables the particle therapy system to treat different depths of the tumor in parallel. Such a particle therapy system selects a subset of the multiple energy levels generated from a target to conform with a treatment plan for the tumor. This helps prevent unnecessary irradiation of healthy tissue adjacent to the tumor.

In disclosed embodiments, a particle therapy system (e.g., system 100) may include an interaction chamber (e.g., interaction chamber 108) configured to contain a target (e.g., target 110). The particle therapy system may also include an energy source (e.g., electromagnetic radiation source 102) configured to generate a pulsed electromagnetic radiation beam (e.g., electromagnetic radiation beam 104) for irradiating the target and to thereby produce a polyenergetic particle beam (e.g., particle beam 112) containing multiple energy levels that span an energy range at least about 5 MeV. The particle therapy system may additionally include a data interface (e.g., data interface 510) configured to receive data associated with a treatment plan for a tumor (e.g., treatment volume 120). The particle therapy system may further include a at least one processor (e.g., control system 122) configured to enable selection of a subset of the multiple energy levels to conform with a treatment plan for the tumor. The particle therapy system may also include a magnetic beamline (e.g., beamline 114) configured to deliver to the tumor a portion of the polyenergetic particle beam associated with the selected subset of multiple energy levels.

Consistent with the present disclosure, particle therapy system 100 may provide the desired energy levels of the charged particles. Particle beam 112 may include charged particles with multiple energy levels spanning at least 5 MeV (e.g., at least 10 MeV, at least 25 MeV, or at least 50 MeV). Consistent with the present disclosure, the term “charged particles with energy levels spanning at least X MeV” means the energies of the charged particles may spread between Y MeV and Y+X MeV or more when the energies of the charged particles are measured at full width at half maximum (FWHM). When it said that particle beam 112 contains multiple energy levels spanning at least about 5 MeV, for example, it means when the charged particles are protons, they may have energies at FWMH ranging from about 60 MeV to 65 MeV (or more than 65 MeV), from about 100 MeV to 105 MeV (or more than 105 MeV), from about 120 MeV to 125 MeV (or more than 125 MeV), and so on. Throughout the specification, the listed example energies may refer to protons. One skilled in the art would recognize that, for ions, the values of the energies may be adjusted. For example, particle beam 112 may include ions with multiple energy levels spanning at least 5 MeV/nucleon (e.g., at least 10 MeV/nucleon, at least 25 MeV/nucleon, or at least 50 MeV/nucleon).

System 100 selects a subset of the multiple energy levels that conforms with a treatment plan for the tumor. As used in this specification, the term “treatment plan” may refer to any information that can be used directly or indirectly to prescribe a particle treatment. For example, in some embodiments, a treatment plan may include data associated with one or more treatment parameters, such as a tumor-related parameter (e.g., the location and size of the tumor), a target-related parameter (e.g., the composition, shape, size, structure, or orientation of the target), a dose, a dose rate, a gantry position, a gantry speed, a collimator position, a beam energy, a beam-on condition, a beam-off condition, a patient support position, or other relevant parameters. The treatment plan may be used to carry out at least a portion of the treatment session, and such information may be derived from one or more treatment parameters, including those listed above.

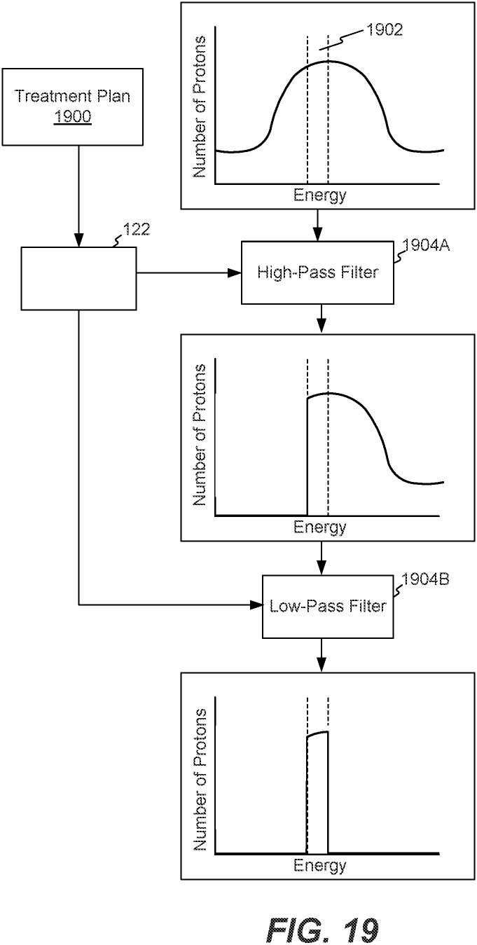

FIG. 19 illustrates an exemplary process for selecting a subset of multiple energy levels (e.g., desired energy levels 1902) that conform with a treatment plan 1900 for the tumor. Desired energy levels 1902 may span more than 0.1 MeV. For example, desired energy levels 1902 may span for more than 2 MeV, more than 4 MeV, more than 5 MeV, or more than 10 MeV. Desired energy levels 1902 may span less than 50 MeV. For example, desired energy levels 1902 may span less than 30 MeV, less than 20 MeV, less than 10 MeV, or less than 5 MeV. In one embodiment, the subset of the multiple energy levels spans about ± 2% of the median of the selected subset of the multiple energy levels. The selection of desired energy levels 1902 may be accomplished by applying one or more band-pass filter to the polyenergetic particle beam (i.e., particle beam 112). Control system 122 may receive treatment plan 1900 and determine desired energy levels 1902 for a given gantry orientation. Thereafter, control system 122 applies an adjustable high-pass filter 1904A to isolate charged particles having energies greater than a first energy cut-off threshold and apply an adjustable low-pass filter 1904B to isolate charged particles having energies less than a second energy cut-off threshold. To achieve particle energy filtering, one or more particle beam adjustment components of beamline 114 may be selectively activated and/or controlled by one or more automated switches, such as a spark switch or photoconductive switch. Selective activation may be governed by control system 122, which may interface with the automated switch and particle beam adjustment components. The automated switch may be activated or deactivated by a signal generated by control system 122. The signal may be generated based on feedback, such as any of the forms of feedback described in the present disclosure or described in U.S. Pat. No. 10,395,881, incorporated here by reference.

As described with reference to FIGS. 10 through 12, electromagnetic radiation source 102 may cause a simultaneous emission of a plurality of charged particles 1332 at differing energy levels each time target 110 is irradiated. In a first example, target 110 may be hydrogen-rich, and charged particles 1332 may be protons. In a second example, target 110 may be carbon-rich and the charged particles may be carbon ions. Upon emission of the plurality of charged particles 1332, beamline 114 may concurrently deliver multiple charged particles 1332 at differing energy levels to different penetration depths along the Z axis of tumor 1008. Specifically, with reference to FIG. 10, a bunch of charged particles 1010B may irradiate the locations of tumor 1008 from Z=1 to 12. Consistent with the present disclosure, beamline 114 may deliver to tumor 1008 a portion of the polyenergetic particle beam 112 that includes charged particles with energy levels in the selected subset of the multiple energy levels. In some embodiments, particle therapy system 100 may prevent delivery of particles with energy levels higher and/or lower than the selected subset of the multiple energy levels, for example, by using low-pass filter 1904B and/or high-pass filter 1904A. In some implementations, preventing the delivery of particles with energy levels higher than the selected subset of the multiple energy levels may include reducing the energy of the particles with the energy levels higher than the selected subset of the multiple energy levels to a level within the selected subset of the multiple energy levels. For example, when desired energy levels 1902 are between about 60 MeV and 250 MeV, particle therapy system 100 may be configured to use an energy degrader to reduce the energy of charged particles from about 250 MeV to 60 MeV.

In some embodiments, beamline 114 may include a beam dump for disposing of charged particles with energy level other than the selected energy band and/or an energy degrader for reducing the energy of the particles with the energy levels higher than the selected subset of the multiple energy levels to a level within the selected subset of the multiple energy levels. The energy degrader may be used to reduce energy and/or flux of the charged particles with energy level other than the selected energy band. To filter particle beam using an energy degrader, charged particles may be diverted through the degrader, where they interact with the degrader. Charged particles transmitted through the degrader along the trajectory of the particle beam then have reduced energies. Other charged particles may either be absorbed by the energy degrader or diverted from the trajectory of the particle beam. An energy degrader may include, for example, carbon, plastics, beryllium, metals such as copper or lead, or any material that is effective at reducing the energy or flux of a particle beam. An energy degrader may also consist of any shape effective at reducing the energy or flux of the particle beam, including a wedge, a double wedge separated by a gap (which may be filled with air or another material), a cylinder, a rectangle, or any other material or configuration capable of degrading the beam.

A conventional Energy Selection System (ESS) may include a dipole bending magnet and a slit. Charged particles passing through the dipole bending magnet are deflected according to their energies, and the slit selects a subset of energies. In one embodiment, system 100 may include a particle separator for stratifying the polyenergetic particle beam 112 into a plurality of sub-beams. The selection of the subset of the multiple energy levels may include using the particle separator to stratify the polyenergetic particle beam 112 into a plurality of sub-beams with desired energy levels. Doing so may enable beamline 114 to concurrently deliver a first part of the plurality of sub-beams to patient 118 and avoid delivering a second part of the plurality of sub-beams to patient 118. In a first embodiment, a particle separator may include at least one dipole magnet and an adjustable, multi-slit array. In a second embodiment, a particle separator may include a focusing magnet and a collimator.

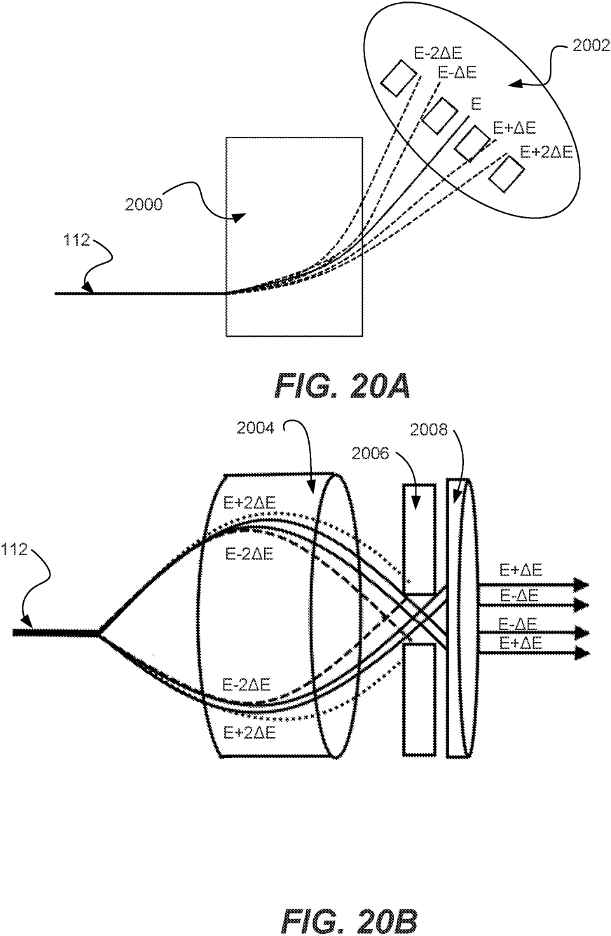

FIG. 20A illustrates a first embodiment of the particle separator. Polyenergetic particle beam 112 may pass through a dipole magnet 2000, which stratifies polyenergetic particle beam 112. The charged particles of the polyenergetic particle beam 112 are diverted based on their energy levels and enter an adjustable, multi-slit array 2002. Each silt in adjustable, multi-slit array 2002 may be associated with a differing energy level, and when the charged particles exit adjustable, multi-slit array 2002, they are arranged in a plurality of substantially monoenergetic sub-beams. In some embodiments, control system 122 may control adjustable, multi-slit array 2002 to select the energy levels of the plurality of sub-beams to be delivered to the tumor. In other embodiments, control system 122 may control adjustable, multi-slit array 2102 to create a volume of particles from different energy levels delivered to the tumor. For example, a transverse slit system or a two-dimensional slit system could be used to reduce the relative flux of particles in each energy level. Thus, beamline 114 may deliver to the tumor a portion of the polyenergetic particle beam 112 associated with less than all of the particles with energy levels in the selected subset of the multiple energy levels. A more sophisticated energy selection system can be designed, combining several dipoles and/or quadrupoles to have a parallel separation of energies at slit level. The basic principle, however, of controlling the multi-slit array to select the desired energy levels would remain the same.

FIG. 20B illustrates a second embodiment of the particle separator. Polyenergetic particle beam 112 may pass through focusing magnet 2004, an adjustable silt 2006, and collimator 2008. Consistent with the present disclosure, beamline 114 may deliver to the tumor a portion of the polyenergetic particle beam 112 associated with desired energy levels 1902. In the illustrated example, desired energy levels 1902 may be E±ΔE, and most of the charged particles with energy more or less than the selected subset of multiple energy levels, such as E±2ΔE, will be blocked by slit 2006. Thereafter, collimator 2008 may direct the plurality of sub-beams with the desired energy levels. The portion of the polyenergetic particle beam associated with desired energy levels 1902 may include at least about 3×10⁶ charged particles per second, for example, at least about 3×10⁷ charged particles per second. In some embodiments, beamline 114 may concurrently deliver to a first tumor location a first group of charged particles associated with a first energy level (e.g., E+0.5ΔE) and deliver to a second tumor location a second group of charged particles associated with a second energy level (e.g., E-0.5ΔE). The first tumor location is deeper than the second tumor location.

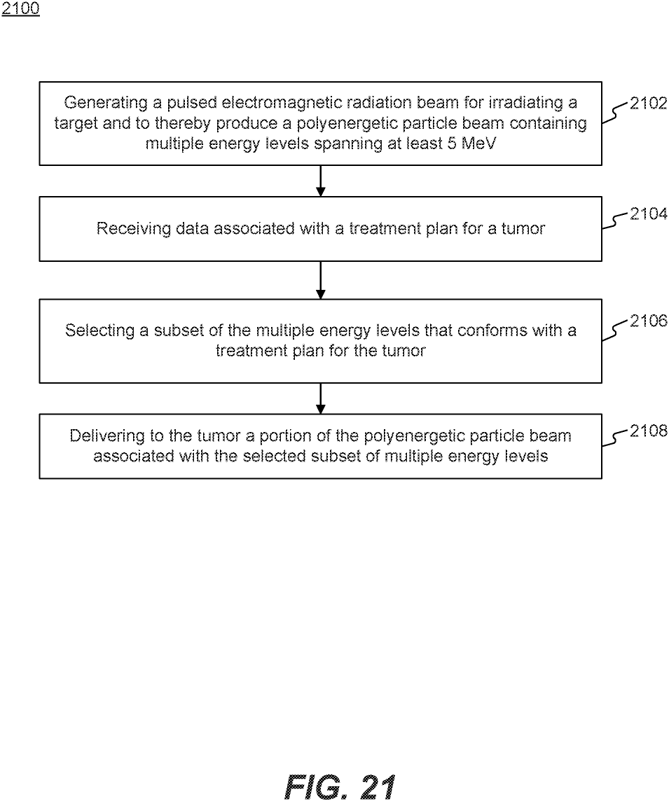

FIG. 21 is a flowchart of an example process 2100 for particle therapy executed by a processing device of control system 122 according to embodiments of the present disclosure. For purposes of illustration, in the following description, reference is made to certain components of particle therapy system 100. It will be appreciated, however, that other implementations are possible and that any combination of components or devices may be utilized to implement the exemplary method. It will also be readily appreciated that the illustrated method can be altered to modify the order of steps, delete steps, or further include additional steps, such as steps directed to optional embodiments.

Disclosed embodiments may include “generating a pulsed electromagnetic radiation beam for irradiating a target and to thereby produce a polyenergetic particle beam containing multiple energy levels spanning at least 5 MeV.” The target may be hydrogen-rich, and the polyenergetic particle beam may include protons. Alternatively, the target may be carbon-rich, and the polyenergetic particle beam may include carbon ions. By way of example only, according to step 2102 in FIG. 21, the processing device may generate pulsed electromagnetic radiation beam 104 for irradiating target 110 and thereby produce a pulsed polyenergetic particle beam 112 containing charged particles with multiple energy levels spanning at least 5 MeV, at least 15 MeV, at least 25 MeV, at least 35 MeV, at least 45 MeV, or at least 50 MeV.

Disclosed embodiments may further include “receiving data associated with a treatment plan for a tumor.” As discussed earlier, the treatment plan may be received using a communications network from a remote source. By way of example only, according to step 2104 in FIG. 21, the processing device may receive data associated with treatment plan 1900 for tumor 1008. The data associated with treatment plan 1900 may be received using data interface 510 and may include values of one or more treatment parameters, including tumor-related parameters (e.g., the location and size of the tumor), target-related parameters (e.g., composition, shape, size, structure, or orientation of the target), a dose, a dose rate, a gantry position, a gantry speed, a collimator position, a beam energy, a beam-on condition, a beam-off condition, or other relevant parameters.

Disclosed embodiments may further include “selecting a subset of the multiple energy levels that conforms with a treatment plan for the tumor.” As discussed above, the subset of the multiple energy levels may contain multiple energy levels spanning between about 0.1 MeV to 50 MeV. By way of example only, according to step 2106 in FIG. 21, the processing device may select a subset of the multiple energy levels that conforms with a treatment plan for tumor 1008. According to some embodiments, the processing device may enable selection of a subset of the multiple energy levels by applying one or more energy filters (e.g., filters 1904) to polyenergetic particle beam 112. According to other embodiments, the processing device may enable the selection of the subset of multiple energy levels by using a particle separator configured to stratify the polyenergetic particle beam into a plurality of sub-beams. Selection of the subset of the multiple energy levels may include stratifying polyenergetic particle beam 112 into a plurality of sub-beams with desired energy levels 1902. Thereafter, at least some of the plurality of sub-beams may be concurrently delivered to the tumor using beamline 114. In a first example, the particle separator may include a focusing magnet (e.g., focusing magnet 2004) and a collimator (e.g., collimator 2008). In a second example, the particle separator may include at least one dipole magnet (e.g., dipole magnet 2000) and an adjustable multi-slit array (e.g., adjustable multi-slit array 2002). The processing device may control the adjustable multi-slit array to select the energy levels of sub-beams delivered to the tumor and/or control the adjustable multi-slit array to affect a volume of particles from different energy levels delivered to the tumor.

Disclosed embodiments may further include “delivering to the tumor a portion of the polyenergetic particle beam associated with the selected subset of multiple energy levels.” As discussed earlier, the electromagnetic radiation source may cause simultaneous emission of a plurality of charged particles at differing energy levels each time the target is irradiated, and the magnetic beamline may be configured to concurrently deliver multiple charged particles at differing energy levels to a treatment volume. For example, using only particles generated concurrently from an irradiation of the target, the magnetic beamline may deliver to a first tumor location a first group of charged particles associated with a first energy level and deliver to a second tumor location a second group of charged particles associated with a second energy level. By way of example only, according to step 2108 in FIG. 21, the processing device may deliver to tumor 1008 a portion of the polyenergetic particle beam 112 associated with the selected subset of multiple energy levels. The portion of the polyenergetic particle beam delivered to the tumor may include at least about 3×10⁶ charged particles per second. According to some embodiments, the processing device may control beamline 114 to deliver to tumor 1008 a portion of the polyenergetic particle beam 112 that includes particles with energy levels in a selected subset of the multiple energy levels. In some cases, the processing device may control beamline 114 to deliver to tumor 1008 a portion of the polyenergetic particle beam 112 associated with less than all of the particles with energy levels in the selected subset of the multiple energy levels. According to some embodiments, the processing device may prevent delivery of particles with energy levels higher than the selected subset of the multiple energy levels. For example, the processing device may control beamline 114 to reduce the energies of particles with the energy levels higher than the selected subset of the multiple energy levels to within the selected subset of the multiple energy levels.

The foregoing description has been presented for purposes of illustration. It is not exhaustive and is not limited to the precise forms or embodiments disclosed. Modifications and adaptations will be apparent to those skilled in the art from consideration of the specification and practice of the disclosed embodiments. Additionally, although aspects of the disclosed embodiments are described as being stored in memory, one skilled in the art will appreciate that these aspects can also be stored on other types of computer-readable media, such as secondary storage devices, for example, hard disks or CD ROM, or other forms of RAM or ROM, USB media, DVD, Blu-ray, other optical drive media, or remote storage locations accessible over a network.

Computer programs based on the written description and disclosed methods are within the skill of an experienced developer. The various programs or program modules can be created using any of the techniques known to one skilled in the art or can be designed in connection with existing software. For example, program sections or program modules can be designed in or by means of.Net Framework,.Net Compact Framework (and related languages, such as Visual Basic, C, etc.), Java, C++, Objective-C, HTML, HTML/AJAX combinations, XML, or HTML with included Java applets.

Moreover, while illustrative embodiments have been described herein, the scope of any and all embodiments having equivalent elements, modifications, omissions, combinations (e.g., of aspects across various embodiments), adaptations and/or alterations as would be appreciated by those skilled in the art based on the present disclosure. The limitations in the claims are to be interpreted broadly based on the language employed in the claims and not limited to examples described in the present specification or during the prosecution of the application. The examples are to be construed as non-exclusive. Further, the steps of the disclosed methods may be modified in any manner, including by reordering steps and/or inserting or deleting steps. It is intended that the specification and examples be considered as illustrative only, with a true scope and spirit being indicated by the following claims and their full scope of equivalents.

1. A particle therapy system, comprising:
an interaction chamber for containing a target;
an electromagnetic radiation source configured to generate a pulsed electromagnetic radiation beam of at least 100 terawatts and at a repetition rate of at least 20 Hz;
optics configured to direct the pulsed electromagnetic radiation beam along a path towards a target in the interaction chamber; and
at least one actuator configured to cause relative movement between the target and the electromagnetic radiation beam at a speed associated with the repetition rate of the electromagnetic radiation source, to thereby vary a location of interaction of the pulsed electromagnetic radiation beam on a surface of the target and thereby cause a resultant emission from the target of at least 3×10⁶ charged particles per pulse.

2. The system of claim 1, wherein the speed is associated with a rate equal to or exceeding the repetition rate of the electromagnetic radiation source.

3. The system of claim 1, wherein the resultant emission includes negatively charged particles for delivery to a patient.

4. The system of claim 1, wherein the resultant emission includes positively charged particles for delivery to a patient.

5. The system of claim 1, wherein the interaction chamber is configured to contain a hydrogen-rich target, and the charged particles are protons.

6. The system of claim 1, wherein the interaction chamber is configured to contain a carbon-rich target, and the charged particles are carbon ions.

7. The system of claim 1, wherein the target is sized to enable at least 100 locations of interaction with the pulsed electromagnetic radiation beam.

8. The system of claim 1, wherein the target includes a plurality of microstructured elements, and each location of interaction includes at least one microstructured element.

9. The system of claim 8, wherein the electromagnetic radiation source is configured to destroy microstructured elements at each differing location of interaction.

10. The system of claim 1, wherein the at least one actuator is configured to cause movement of the target within the interaction chamber.

11. The system of claim 10, wherein the interaction chamber includes a target stage for supporting the target, and the at least one actuator is configured to rotate the target stage at a speed of at least 0.5 RPM.

12. The system of claim 10, wherein the interaction chamber includes a target stage for supporting the target, and the at least one actuator is configured to linearly move the target stage by at least 20 mm/s.

13. The system of claim 1, wherein the at least one actuator is configured to rotate the target.

14. The system of claim 1, further comprising a processor configured to cause a change in the path of the electromagnetic radiation beam.

15. The system of claim 14, wherein the optics includes an adjustable mirror, and the at least one actuator is configured to vary the adjustable mirror.

16. The system of claim 14, wherein the optics include adaptive optics.

17. The system of claim 1, wherein the at least one actuator includes a first actuator configured to cause movement of the target within the interaction chamber and a second actuator configured to cause a change in the path of the electromagnetic radiation beam.

18. A method for particle therapy, comprising:
generating a pulsed electromagnetic radiation beam of at least 100 terawatts and at a repetition rate of at least 20 Hz;
directing the pulsed electromagnetic radiation beam along a path towards a target in the interaction chamber; and
causing a relative movement between the target and the electromagnetic radiation beam at a speed associated with the repetition rate of the electromagnetic radiation source, to thereby vary a location of interaction of the pulsed electromagnetic radiation beam on the surface of target and thereby cause a resultant emission from the target of at least 3×10⁶ charged particles per pulse.

19. The method of claim 18, wherein:
the pulsed electromagnetic radiation beam includes a plurality of pulse chains, each pulse chain including a preliminary pulse and a main pulse;
the preliminary pulse exceeds an energy flux threshold and has an energy flux between 0.1 and 10 J/cm² at the target; and
the main pulse has an intensity of at least 10¹⁸ W/cm² at the target.

20. The method of claim 19, wherein a time separation between the preliminary pulse and the main pulse is between 1 ns and 26 ns, such that during the time separation the target is free from irradiation exceeding the energy flux threshold.

21-100. (canceled)
